(19) United States
(12) Patent Application Publication
(10) Pub. No.: US 2009/0123934 A1
(43) Pub. Date: May 14, 2009

(54) Inhibition of polo kinase by matrimony maintains G2 arrest in the meiotic cell cycle

(76) Inventors: Youbin Xiang, Overland Park, KS (US); Sue Jaspersen, Lenexa, KS (US); Laurence Florens, Leawood, KS (US); Sarah Kendall Smith, Kansas City, MO (US); R. Scott Hawley, Overland Park, KS (US)

Correspondence Address: Kevin C. Hooper;BRYAN CAVE LLP, 1290 Avenue of the Americas, New York, NY, 10104, US

(21) Appl. No.: 12/288,322

(22) Filed: Oct. 17, 2008

Related U.S. Application Data
(60) Provisional application No. 60/999,447, filed on Oct. 18, 2007.

(51) Int. Cl. C12Q 1/68; C12N 9/99; C12N 5/06; G01N 33/53; C12Q 1/48

(52) U.S. Cl. 435/6; 435/184; 435/375; 435/15; 435/7.1

(57) ABSTRACT

Matrimony (Mtrm) acts as a negative regulator of Polo kinase (Polo) during the later stages of G2 arrest. Indeed, both the repression of Polo expression until stage 11 and the inactivation of newly synthesized Polo by Mtrm until stage 13 play critical roles in maintaining and properly terminating G2 arrest. Our data suggest a model in which the eventual activation of Cdc25 by an excess of Polo at stage 13 triggers NEB and entry into prometaphase. In view of the foregoing, methods for modulating oocyte maturation are provided. More particularly, methods are provided for in vitro maturation of an oocyte. Further provided are methods for identifying functional orthologs of a Drosophila Matrimony polypeptide, as well as inhibitors thereof.

### RELATED APPLICATIONS

This application is based upon and claims the benefit of priority from U.S. Provisional Application No. 60/999,447, filed Oct. 18, 2007, the entire contents of which are incorporated by reference as if recited in full herein.

### FIELD OF THE INVENTION

The present invention relates to methods for modulating oocyte maturation, including methods for in vitro maturation of an oocyte. The present invention also relates to methods for identifying functional orthologs of a Drosophila Matrimony polypeptide, as well as to methods for identifying inhibitors of such orthologs.

### BACKGROUND OF THE INVENTION

Many meiotic systems in animal females include a lengthy arrest in G2 that separates the end of pachytene from nuclear envelope breakdown (NEB). However, the mechanisms by which a meiotic cell can arrest for long periods of time (decades in human females) have remained a mystery. One can imagine that both the maintenance and the termination of this arrest might involve either or both of two mechanisms—the transcriptional or translational repression of a protein that induces NEB, and thus meiotic entry, or the presence of an inhibitory protein that precludes entry into the first meiotic division. Because Drosophila females exhibit a prolonged G2 arrest (see FIG. 1) and are amenable to both genetic and cytological analyses, they provide an ideal system in which to study this problem.

The ovaries of Drosophila females are comprised of a bundle of ovarioles, each of which contains a number of oocytes arranged in order of their developmental stages [1-3]. For our purposes, the process of oogenesis may be said to consist of three separate sets of divisions: the initial stem cell divisions, which create primary cystoblasts; four incomplete cystoblast divisions, which create a 16 cell cyst that contains the oocyte; and the two meiotic divisions. Although a great deal is known regarding the mechanisms that control cystoblast divisions and oocyte differentiation, relatively little is known about the mechanisms by which the progression of meiosis is controlled.

As is the case in many meiotic systems, female meiosis in Drosophila involves pre-programmed developmental pauses. The two most prominent pauses during Drosophila meiosis are an arrest that separates the end of pachytene at stages 5-6 from NEB at stage 13, and a second pause that begins with metaphase I arrest at stage 14 and continues until the egg passes through the oviduct. It is the release of this second pre-programmed arrest event that initiates anaphase I and allows the completion of meiosis I followed by meiosis II. As shown in FIG. 1, the end of meiotic prophase by dissolution of the synaptonemal complex (SC) at stages 5-6 [4,5], is separated from the beginning of the meiotic divisions, defined by NEB at stage 13, by approximately 40 hours to allow for oocyte growth.

In view of the foregoing, it would be advantageous to identify mechanisms, molecules, and methods for understanding and modulating meiotic cell arrest in, e.g., G2.

### SUMMARY OF THE INVENTION

The present invention is directed to achieving these and other goals. Thus, one embodiment of the present invention is a method for modulating oocyte maturation. This method includes the step of contacting an oocyte with an amount of a molecule selected from the group consisting of Polo kinase (Polo), an ortholog of Polo, a modulator of Polo or its ortholog, and combinations thereof, which amount is sufficient to achieve modulation of oocyte maturation.

Another embodiment of the present invention is a method for in vitro maturation of an oocyte. This method includes the step of culturing an oocyte in a suitable media comprising at least one component that triggers nuclear envelope breakdown and/or entry into prometaphase.

A further embodiment of the present invention is a method for preserving oocytes obtained from a patient prior to undergoing a therapy that may damage or destroy the patient's ovaries, such as, for example, chemo- or radiation therapy. This method includes the steps of (a) obtaining an oocyte from an ovary of the patient, (b) culturing the oocyte in a suitable media including at least one component that triggers oocyte maturation, and (c) preserving, such as, e.g., cryopreserving the matured oocyte.

An additional embodiment of the present invention is a method for identifying a functional ortholog of a Drosophila Matrimony polypeptide. This method includes the steps of (a) screening polypeptides from an oocyte preparation for their ability to interact with Polo kinase (Polo) or an ortholog thereof and (b) identifying which, if any, of the polypeptides screened in step (a) act as an inhibitor of Polo or an ortholog thereof.

A further embodiment of the present invention is a method for identifying a candidate compound that may be effective to inhibit an ortholog of Drosophila Matrimony (Mtrm). This method includes the steps of (a) contacting a test oocyte that expresses a functional ortholog of a Drosophila Matrimony polypeptide identified in a functional ortholog assay disclosed herein with a candidate compound and (b) determining whether the candidate compound causes a decrease in Mtrm function, an increase in Polo kinase function, nuclear envelop break down, and/or entry into prometaphase 1, wherein a candidate compound that decreases Mtrm function, increases Polo kinase function, triggers nuclear envelop break down (NEB) and/or entry into prometaphase 1 relative to a control cell that is not contacted with the candidate compound is indicative that the candidate compound may be effective to inhibit the ortholog of Drosophila Mtrm.

Another embodiment of the invention is a method for identifying a candidate compound that modulates the binding of Matrimony or an ortholog thereof to Polo or an ortholog thereof. This method comprises the steps of: (a) contacting Matrimony or an ortholog thereof with Polo or an ortholog thereof under conditions suitable to form a Matrimony-Polo complex; (b) contacting the Matrimony-Polo complex with a candidate compound; and (c) determining the ability of the candidate compound to modulate binding of Matrimony or an ortholog thereof to Polo or an ortholog thereof, wherein modulation of the binding of Matrimony or an ortholog thereof to Polo or an ortholog thereof indicates that the candidate compound is effective to modulate the binding of Matrimony or ortholog thereof to Polo or an ortholog thereof.

Another embodiment of the invention is a method for identifying a functional ortholog of a Drosophila Matrimony polypeptide. This method comprises: (a) screening polypeptides from an oocyte preparation for their ability to interact with Polo kinase (Polo) or an ortholog thereof; and (b) identifying which, if any, of the polypeptides screened in step (a) act as an inhibitor of Polo or an ortholog thereof.

### BRIEF DESCRIPTION OF THE FIGURES

The application contains at least one drawing executed in color. Copies of this patent and/or application publication with color drawing(s) will be provided by the Office upon request and payment of the necessary fee. The following drawings form part of the present specification and are included to further demonstrate certain aspects of the present invention. The invention may be better understood by reference to one or more of these drawings in combination with the Detailed Description and the Examples presented herein.

FIG. 1 is a schematic depiction of oocyte development in Drosophila melanogaster showing the timing (in hours) of the relevant stages. The end of meiotic prophase, as defined by SC dissolution, occurs at stages 5-6. By the end of stages 5-6, the chromosomes have condensed into a dense mass known as the karyosome, as pointed out by Mahowald and Kambysellis [2]. The karyosome remains compacted until stages 8-10, at which time it de-condenses and a high level of transcription is observed. The chromosomes re-compact during stages 11 and 12 to form a tight mass that is released into the cytoplasm upon nuclear envelope breakdown (NEB) at stage 13. The end of pachytene is separated from NEB by approximately 40 hours.

FIG. 2 shows the mtrm gene and its expression pattern. FIG. 2A is a schematic diagram of the 651-bp mtrm gene. The mtrm¹²⁶ deletion allele, which was created by imprecise excision of the P element insertion mutation KG08051, is deleted for 203 bases (80 bases upstream of the first ATG in mtrm and 123 downstream of that ATG). FIG. 2B shows a Western blot analysis using an anti-Mtrm antibody of protein extracts from the indicated tissues. These experiments reveal that Mtrm, a 27 kDa protein, is expressed only in ovaries. The lower panel displays a Western blot of equal amounts of protein from the same extracts probed with antibody to alpha-tubulin (50 kDa MW). FIG. 2C shows immunostaining using the anti-Mtrm antibody to stage 9 oocytes, revealing that Mtrm is expressed in the nuclei of both oocytes and nurse cells in wild-type egg chambers, but not in mtrm homozygote egg chambers. This latter finding indicates the anti-Mtrm antibody is specific to Mtrm. FIG. 2D shows the timing of Mtrm expression during oocyte development. Endogenous Mtrm expression is not detectable before stage 5. At stage 5, Mtrm localizes to both the oocyte and nurse cells. (Scale −30 μm.)

FIG. 3 shows that reducing the dose of polo⁺ suppresses mtrm defects and increasing the dose of polo⁺ partially mimics the effects of mtrm. FIG. 3A is a schematic diagram of the polo gene (black boxes depict the five exons) indicating the insertions sites for the two polo alleles (polo¹⁶⁻¹ and polo^{KG03033}). FIG. 3B is a summary of the genetic interaction of mtrm and polo mutants as examined by assaying the frequency of nondisjunction of the X and 4^{th} chromosomes. As shown by Harris et al. [9], mtrm/+ heterozygotes display high levels of nondisjunction for both achiasmate X and 4^{th} chromosomes (42% and 37%, respectively) when compared to mtrm⁺/mtrm⁺ females. However, simultaneously reducing the dose of polo, as a result of heterozygosity for either the two P-element insertion site mutants or a deficiency that uncovers polo (Df(3L)rdgC-co2), suppresses the meiotic phenotype of mtrm/+ heterozygotes. FIG. 3C shows that expression of the UASP-polo⁺ transgene in mtrm⁺/mtrm⁺ females results in a dosage-dependent increase in the frequency of achiasmate nondisjunction for both the X and the 4^{th} chromosomes. However, two weaker alleles of polo, polo⁰¹⁶⁷³ and polo¹, showed little or no suppression of the segregational defect (data not shown). The polo¹ mutant, which is the weakest of the known polo mutants (it is viable over a deletion), is the result of a point mutation at base pair 725, V242E, in the kinase domain. Although polo⁰¹⁶⁷³ is recessive lethal, it must retain some degree of function because it complements at least one other hypomorphic allele of polo, polo^{x8}. The results indicate that reduction of polo⁺ dosage rescues mtrm defects and the suppressive effect of a given polo mutant correlates with the severity in the reduction of Polo function.

FIG. 4 shows that Mtrm physically interacts with Polo with a stoichiometry of approximately 1:1. FIG. 4A is a schematic depiction of the Mtrm protein. Mtrm has two potential PBD binding sites, STP and SSP, with the central serine/threonine residue at 40 and 124, respectively, and a SAM domain at the C-terminus. Two independent transgenes expressing mutated PBD binding sites were generated: Mtrm^{T(40)A}, which disrupts the STP site and Mtrm^{S(124)A}, which disrupts the SSP site. FIG. 4B shows the results of co-immunoprecipitation experiments demonstrating that Mtrm and Polo physically interact. An anti-Mtrm antibody precipitates Polo from wild-type ovary extracts (lane 1). Expression of the mutated PBD binding site constructs in a mtrm null background reveals that Mtrm^{S(124)A} does not ablate the Mtrm-Polo interaction (lane 2). However, Mtrm^{T(40)A} failed to bind Polo (lane 3) indicating that the STP motif is critical for the Mtrm-Polo interaction. FIG. 4D shows the results of a MudPIT mass spectrometry assay using three independent affinity purifications from ovarian extracts expressing a C-terminally 3×FLAG-tagged Mtrm. Among the reproducible and significant (p value<0.001) proteins identified in all three analyses, Polo was detected by multiple peptides and stood out as the only protein recovered at levels similar to those of Mtrm, as estimated by normalized spectral counts (NSAF). FIG. 4E shows phosphorylated sites detected in Mtrm (blue bars) and Polo (yellow bar). Modification levels were estimated based on local spectral count and averaged across the three immunoprecipitations. The numbers in each bar represent the number of times (out of 3) the residues were found modified. The STP site required for Polo binding is also required for Mtrm function. As noted above, FM7/X; mtrm/+ heterozygotes display approximately 40% X ND and 37% 4^{th} nondisjunction. Although the Mtrm^{S(124)A} protein was able to rescue the meiotic defect (3.6% X and 4.4% 4^{th} ND), the Mtrm^{T(40)A} protein displayed similar levels of nondisjunction as mtrm/+ heterozygotes, indicating that the STP motif is critical for Mtrm function (FIG. 4C). The finding that only the STP site is required for both Mtrm function and the binding of Mtrm to Polo is consistent with the observation that only the STP motif is conserved across all twelve sequenced Drosophila genomes, while the SSP motif is conserved only within the six species that belong to the D. melanogaster-D. ananassae clade.

FIG. 5 shows that mtrm causes precocious NEB. FIG. 5A shows representative examples of NEB in stage 11 and 12 egg chambers for wild-type (w¹¹¹⁸) and mtrm¹²⁶ homozygotes. NEB in wild-type oocytes occurs at stage 13. The nucleus is still present (seen as a dark mass by phase contrast microscopy) at stage 11 and stage 12 in wild-type. mtrm homozygotes show precocious NEB (absence of the dark mass) that can occur prior to stage 11. (Scale—60 μm.) FIG. 5B is a summary of NEB in stage 11 and stage 12 egg chambers for wild-type (w¹¹¹⁸) (mtrm⁺/mtrm⁺), mtrm heterozygotes (mtrm¹²⁶/+), mtrm homozygotes (mtrm¹²⁶/mtrm¹²⁶), and double heterozygotes for both mtrm, polo (mtrm¹²⁶+/+polo¹⁶⁻¹).

FIG. 6 shows that mtrm is defective in karyosome maturation before NEB. FIG. 6A shows representative examples of karyosomes 12-16 minutes before and at NEB for wild-type (X/X), mtrm¹²⁶/mtrm¹²⁶ (X/X,), mtrm¹²⁶/mtrm⁺, and mtrm¹²⁶ polo⁺/mtrm⁺ polo¹⁶⁻¹ with achiasmate X chromosomes (FM7/X). The karyosomes in stage 11-12 oocytes, which have a nuclear envelope, were imaged after the injection of Oli-green and Rhodamine-tubulin until NEB. NEB was defined as the time when the nuclear envelope seems ruffled and the Rhodamine-tubulin enters the nucleus. Wild-type displays a circular karyosome with a smooth outline for 12-16 minutes before NEB, whereas mtrm¹²⁶/mtrm⁺ oocytes bear scabrous or bi-lobed karyosomes. The disordered morphology of karyosomes in mtrm¹²⁶/mtrm⁺ oocytes was suppressed by simultaneously reducing the dose of polo. (Scale—5 μm.) Thus, mtrm is defective in karyosome maturation before NEB. FIG. 6B is a summary of karyosome morphology during the 20 min before NEB.

FIG. 7 shows that mtrm causes the individualization of bivalents after NEB. Stage 12 oocytes were injected with Oli-green to visualize karyosomes. Following this injection, we analyzed the change in karyosome structure during NEB using live imaging. Time frames from NEB (time 0) are shown for: FIG. 7A—FM7/X; mtrm⁺/mtrm⁺ for control, FIG. 7B—FM7/X, mtrm¹²⁶/mtrm⁺; and FIG. 7C—FM7/X; mtrm¹²⁶ polo⁺/mtrm⁺ polo¹⁶⁻¹ oocytes. In control oocytes, the karyosome stays condensed after NEB and then becomes elongated at about 13 minutes, presumably as a consequence of the chromosomes establishing proper centromere co-orientation. Almost all control oocytes (8/9) exhibited a karyosome in which chromosomes are tightly associated. In the remaining case, three bivalents could be distinguished but were still physically associated. However, in FM7/X; mtrm¹²⁶/mtrm⁺ oocytes, the 4^{th} chromosomes are separated from a single mass of chromatin at 6-8 minutes after NEB, and then X, 2^{nd} and 3^{rd} chromosomes start to spread out. At approximately 16 minutes after NEB, the chromosomes are individualized into three obvious and fully separate bivalents. The individualized chromosomes begin to re-condense around 46 minutes and form a single mass. Indeed, the majority (11/15) of those oocytes that underwent bivalent individualization eventually formed bipolar spindles with the chiasmate chromosomes properly balanced on the metaphase plate (see also FIG. 4 of Harris et al. 2003 [9]). Thus the karyosome maintenance defect induced by heterozygosity for mtrm does not permanently impair the progression of prometaphase. Additionally, the karyosome maintenance induced by heterozygosity for mtrm was suppressed by reducing the dosage of the polo⁺ gene. As shown below, 10 of the 13 FM7/X; mtrm¹²⁶ polo⁺/mtrm⁺ polo¹⁶⁻¹ oocytes maintained the karyosome as a single mass throughout the process of spindle assembly. The three remaining cases may be described as follows: 1) the karyosome dissolved into three clearly distinguishable bivalents, but this oocyte never succeeded in forming a bipolar spindle; 2) the three major bivalents could be distinguished but did not physically separate; and 3) in an oocyte which may have been leaking or damaged, the bivalents individualized at about eight minutes after the initiation of spindle assembly, but their morphology was abnormally stretched and thread-like. Seven minutes later these chromosomes began to fragment into much smaller pieces which led to the assembly of a spindle with at least five and possibly more poles. It is likely that this case reflects simply the fragility of the karyosome, even in polo¹⁶⁻¹/polo⁺ suppressed oocytes, rather than the defect observed in FM7/X; mtrm¹²⁶/mtrm oocytes that are wild-type for polo.

FIG. 8 shows that heterozygosity for mtrm¹²⁶ impairs the proper co-orientation of achiasmate centromeres during prometaphase. FIG. 8A shows a FISH analysis using probes homologous to the X and 4^{th} chromosomal heterochromatin [29] to assay centromere co-orientation during meiotic prometaphase. In mtrm⁺/mtrm⁺ oocytes carrying either chiasmate X chromosomes (XX females) or achiasmate X chromosomes (FM7/X females), the centromeres of both the X and the 4^{th} are virtually always oriented toward opposite poles (see panels 1 and 4 and FIG. 8B). However, in mtrm/+ heterozygotes the centromeres of achiasmate bivalents are often oriented towards the same pole (see panels 2, 5 and FIG. 8B). In double heterozygotes for both mtrm and polo these defects in achiasmate chromosome centromere co-orientation are greatly suppressed (panels 3 and 6). Thus, heterozygosity for mtrm¹²⁶ impairs the proper co-orientation of achiasmate centromeres during prometaphase. FIG. 8B is a quantitative summary of centromere co-orientation patterns for the various genotypes studied. Although heterozygosity for mtrm¹²⁶ has a dramatic effect on 4^{th} chromosome centromere mal-orientation in both XX and FM7/X females, there is little effect on X chromosome segregation in XX oocytes when compared to the dramatic effect observed in FM7/X females. This is expected based on the genetic studies of Harris et al. [9] who observed that only achiasmate bivalents nondisjoin in mtrm/+ females.

FIG. 9 shows that mutants in the Drosophila cdc25 homolog twine fail to undergo nuclear envelope breakdown in stage 13. FIG. 9A shows representative examples of NEB in stage 13 and 14 egg chambers for wild-type (w¹¹¹⁸) (twe⁺/twe⁺) and twine (twe¹) homozygotes. The nucleus is present (seen as a dark mass by phase contrast microscopy) at early stage 13 but not at late stage 13 and stage 14 in wild-type. twe1 homozygotes show delayed NEB and that the nucleus is still present until early stage 14. (Scale—60 μm.) Thus, mutants in the Drosophila cdc25 homolog twine fail to undergo nuclear envelope breakdown in stage 13. FIG. 9B is a summary of NEB in stage 13 and stage 14 egg chambers for wild-type (w¹¹¹⁸) (twe⁺/twe⁺) and twe¹ homozygotes (twe¹/twe¹).

FIG. 10 shows a model for the control of NEB by Mtrm-induced inhibition of Polo. According to this model, in wild-type Drosophila oocytes the excess of Mtrm inhibits those Polo proteins that are deposited in the oocyte during stages 11 to 12. However, by stage 13 the excess of Polo exceeds the available amount of inhibitory Mtrm proteins. The unencumbered Polo then serves to activate Cdc25, initiating the chain of events that lead to NEB and the initiation of prometaphase. In the absence of a sufficient amount of Mtrm, an excess of functional Polo causes the precocious activation of Cdc25 and thus an early G2/M transition. Based on this model, it appears that decreasing the dose of Mtrm or increasing the dosage of Polo will hasten NEB, while simultaneous reduction in the level of both proteins will normalize the timing of NEB.

FIG. 11 shows expression of mtrm and polo in the later stages of oogenesis. Formaldehyde-fixed egg chambers in wild type, w¹¹¹⁸ were used for co-immunolocalization of Mtrm and Polo with the polyclonal anti-Mtrm antibody from a guinea pig and the monoclonal anti-Polo antibody from mouse. The Mtrm signal is green and the Polo signal is red. As shown above, in stages 4-10 Mtrm is mainly localized in the nuclei of both oocytes and nurse cells. However, in stages 10-12, Mtrm is present in high quantities in the oocyte cytoplasm as well. However, the quantity of Mtrm decreases markedly at stage 13. Polo expression begins at stages 11-12 and is maximal by stage 13. However, Polo is localized in cytoplasm of oocytes and is not abundant in the oocyte nucleus. (‘GV’ indicates germinal vesicle of the oocyte. Scale—40 μm.)

FIG. 12 shows mtrm co-immunoprecipitates with polo using antibodies directed against polo. Mtrm co-immunoprecipitation with GFP-Polo with an anti-GFP antibody, using ovary extracts of GFP-Polo flies (lane 1) and Mtrm co-immunoprecipitation with Polo with an anti-Polo antibody using ovary extracts of wild-type (w¹¹¹⁸) flies (lane 2).

FIG. 13 shows a proposed model for the maintenance of the G2/M arrest in Drosophila female meiosis (49). Stoichiometric (See FIGS. 4D, 4E) inhibition of Polo kinase by Mtrm allows for proper timing of NEB. (Oocytes heterozygous or homozygous for a null allele of mtrm exhibit dosage-dependent precocious NEB (48).

FIG. 14 shows that Plk1 contains a C-terminal PBD, which preferentially binds phospho-threonine/serine residues located in the consensus motif (S-pS/pT-P/X) residing in target proteins (FIG. 14A) (19). Mtrm contains a PBD-binding site, a STP with the central threonine at position 40. Mtrm also contains a SAM domain (FIG. 14B).

FIG. 15 shows a protein sequence alignment of Mtrm homologs from the 12 sequenced Drosophila species, which identifies residues that are potentially critical for Mtrm function. The STP site is embedded within a larger region that is absolutely conserved within the Drosophila genus. Within that region lie two serines, MtrmS48 and MtrmS52, which were found reproducibly phosphorylated while in complex with Polo (FIG. 4D, E) and which fall within a consensus motif for GSK-3 phosphorylation (pS/pT-X—X—X-pS), where the first residue is the site of phosphorylation and the last residue is the priming event. MtrmS66 fits a phosphorylation motif for cyclin B-Cdk1 (pS/pT-P—X—R/K), and MtrmS137 falls within a consensus motif for Polo phosphorylation (D/E-X-pS/pT-Ø-X-D/E). The C-terminal SAM domain is also evolutionarily conserved, as is a region immediately adjacent to it.

FIG. 16 is a schematic of the 217 amino acid Mtrm protein (see, e.g., SEQ ID NO:13). Residues designated by an asterisk were mutated to nonphosphorylatable alanine in various mutant versions of the protein. Residues/regions highlighted yellow appear to be important to the Mtrm-Polo interaction.

FIG. 17 shows the results of a yeast two-hybrid experiment from diploids co-expressing either a wildtype or mutant MtrmAD-fusion protein and a wildtype PoloBD-fusion protein. Serial 10-fold dilutions were plated and incubated for 120 hrs at 30° C. Surprisingly, the MtrmT(40)A mutant is able to interact with Polo kinase in the Y2H system, albeit weakly. Mtrm mutants containing S(48)A and/or S(52)A ablate the interaction. Additionally, deletion of the C-terminal SAM domain in Mtrm impairs Mtrm's ability to interact with Polo kinase.

FIG. 18 is a Western blot showing that Mtrm binds Polo in vitro. Lane 1: Flag-Mtrm incubated in vitro with HA-Polo, then co-immunoprecipitated with anti-Mtrm Ab; Lane 2: HA-polo is immunoprecipitated with anti-Mtrm Ab (as a control).

FIG. 19 is a Western blot showing that Mtrm binds Polo in insect cells (sf9). Lane 1: Flag-Mtrm is expressed in sf9 cells; Lane 2: HA-Polo is expressed in sf9 cells; Lane 3: Both Flag-Mtrm and HA-Polo are co-expressed in sf9 cells.

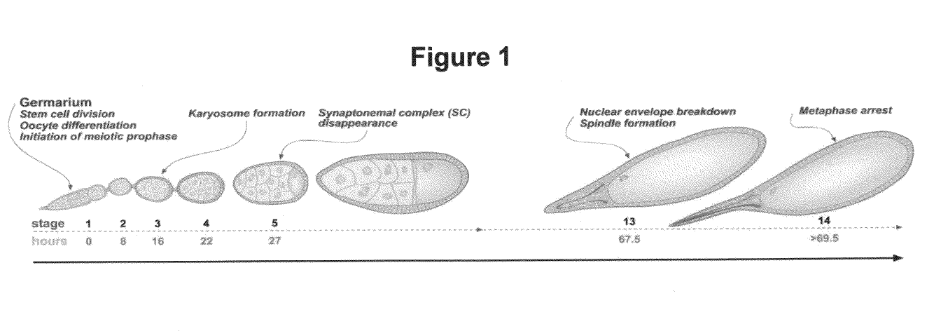

### DETAILED DESCRIPTION OF THE INVENTION

One embodiment of the present invention is a method for modulating oocyte maturation. This method includes the step of contacting an oocyte with an amount of a molecule selected from the group consisting of Polo kinase (Polo), an ortholog of Polo, a modulator of Polo or its ortholog, and combinations thereof, which amount is sufficient to achieve modulation of oocyte maturation.

The Polo ortholog may be a human ortholog. Likewise, the modulator of Polo may be an ortholog of a Mtrm polypeptide. In the present invention, the term “ortholog” denotes a polypeptide or protein obtained from one species that is a functional counterpart of a polypeptide or protein from a different species. Sequence differences among orthologs are the result of, e.g., speciation.

In this embodiment, modulation of oocyte maturation includes activating oocyte maturation. In the present invention, activating oocyte maturation includes contacting the oocyte with an amount of Polo or an ortholog thereof sufficient to initiate nuclear envelope breakdown. Activating oocyte maturation also includes contacting the oocyte with an amount of an inhibitor of Mtrm or an ortholog thereof, which is sufficient to initiate nuclear envelope breakdown.

Alternatively, modulation of oocyte maturation includes inhibiting initiation of nuclear envelope breakdown. In this embodiment, inhibiting oocyte maturation includes contacting the oocyte with an amount of Mtrm or an ortholog thereof sufficient to inhibit initiation of nuclear envelope breakdown. Inhibiting oocyte maturation may also include contacting the oocyte with an amount of an inhibitor of Polo or an ortholog thereof, which is sufficient to inhibit initiation of nuclear envelope breakdown. Non-limiting examples of such an inhibitor include HMN-214 ((E)-4-[2-[2-(p-methoxybenzenesulfonamide)-phenyl]ethenyl]pyridine-1-oxide, Nippon Shinyaku), ON-01910 (a small-molecule benzyl styryl sulfone polo-like kinase 1 inhibitor, Onconova), CYC800 (a small-molecule polo-like kinase-1 (Plk-1) inhibitor, Cyclacel), a signal inhibitor against Plk-1 (Rexahn), Bl-2536 (a polo-like kinase 1 inhibitor, Boehringer Ingelheim), GSK-461364A (a thiophene amide polo-like kinase-1 (Plk) inhibitor, GlaxoSmithKline), PIKT inhibitors (Kiadis), PLK-1 inhibitors (Onconova), PLK-1 inhibitors (Sareum), and combinations thereof.

Another embodiment of the present invention is a method for in vitro maturation of an oocyte. This method includes the step of culturing an oocyte in a suitable media comprising at least one component that triggers nuclear envelope breakdown and/or entry into prometaphase.

In this embodiment, the at least one component is an inhibitor of Matrimony or an inhibitor of a Matrimony ortholog. Preferably, the inhibitor of Matrimony or an inhibitor of a Matrimony ortholog is selected from the group including nucleic acids, polypeptides, polysaccharides, small organic or inorganic molecules, and combinations thereof. For example, the inhibitor is selected from the group including a fusion protein, an antibody, antibody mimetic, domain antibody, targeted aptamer, RNAi, siRNA, shRNA, antisense sequence, small molecule, and combinations thereof.

Preferably, the at least one component is Polo kinase (Polo) or an ortholog thereof.

A further embodiment of the present invention is a method for preserving oocytes obtained from a patient prior to undergoing a therapy that may damage or destroy the patient's ovaries, such as, e.g., chemo- or radiation therapy to treat, e.g., cancer. This method includes the steps of (a) obtaining an oocyte from an ovary of the patient, (b) culturing the oocyte in a suitable media including at least one component that triggers oocyte maturation, and (c) preserving, such as, e.g., cryopreserving the matured oocyte.

The at least one component may be an inhibitor of Matrimony or an inhibitor of a Matrimony ortholog. Preferably, the at least one component is an inhibitor of an ortholog of Drosophila Matrimony identified by an assay of the present invention.

This method may include an additional step of administering the matured oocyte from step (c) to the patient after the therapy, at a time when the patient desires to become pregnant.

A further method of the invention is a method for identifying a candidate compound that modulates the binding of Matrimony or an ortholog thereof to Polo or an ortholog thereof. This method comprises the steps of: (a) contacting Matrimony or an ortholog thereof with Polo or an ortholog thereof under conditions suitable to form a Matrimony-Polo complex; (b) contacting the Matrimony-Polo complex with a candidate compound; and (c) determining the ability of the candidate compound to modulate binding of Matrimony or an ortholog thereof to Polo or an ortholog thereof, wherein modulation of the binding of Matrimony or an ortholog thereof to Polo or an ortholog thereof indicates that the candidate compound is effective to modulate the binding of Matrimony or ortholog thereof to Polo or an ortholog thereof.

In this method, the candidate compound may increase the binding of Matrimony or an ortholog thereof to Polo or an ortholog thereof. In another aspect of this method, the candidate compound may decrease the binding of Matrimony or an ortholog thereof to Polo or an ortholog thereof.

In this method, the candidate compound is selected from the group consisting of a nucleic acid, a polypeptide, a polysaccharide, a small organic or inorganic molecule, and combinations thereof. In another aspect of this method, the candidate compound is selected from the group consisting of a fusion protein, an antibody, an antibody mimetic, a domain antibody, a targeted aptamer, a RNAi, a siRNA, a shRNA, an antisense sequence, a small molecule, and combinations thereof.

With respect to this method, any known binding method/assay may be used so long as it is able to provide a readout, which is suitable to detect whether the candidate compound modulates the binding of Matrimony or an ortholog thereof to Polo or an ortholog thereof. For example, the binding may be determined using a method selected from the group consisting of a yeast two-hybrid (Y2H) assay, a fluorescence resonance energy transfer (FRET) assay, a bioluminescence resonance energy transfer (BRET) assay, a co-immunoprecipitation assay, a label transfer assay, a pull down assay, a tandem affinity purification (TAP) assay, an in vivo crosslinking assay, a chemical crosslinking assay, and a quantitative immunoprecipitation combined with knockdown (QUICK) assay. Preferably, the binding is determined using a yeast two-hybrid assay.

An additional embodiment of the present invention is a method (or assay) for identifying a functional ortholog of a Drosophila Matrimony polypeptide. This method includes the steps of (a) screening polypeptides from an oocyte preparation for their ability to interact with Polo kinase (Polo) or an ortholog thereof and (b) identifying which, if any, of the polypeptides screened in step (a) act as an inhibitor of Polo or an ortholog thereof.

Preferably, the oocyte preparation is obtained from a mammal, such as for example, from a human.

Preferably, the screening step includes an assay selected from the group including yeast two-hybrid (Y2H), fluorescence resonance energy transfer (FRET), bioluminescence resonance energy transfer (BRET), co-immunoprecipitation, label transfer, pull down, tandem affinity purification (TAP), in vivo crosslinking, chemical crosslinking, and quantitative immunoprecipitation combined with knockdown (QUICK) or any other equivalent assay for determining protein-protein interaction.

Preferably, a polypeptide identified in step (b) includes a Polo binding domain (PBD) having amino acids STP, SSP, or both STP and SSP.

Another embodiment of the present invention is a method for identifying a candidate compound that may be effective to inhibit an ortholog of Drosophila Matrimony (Mtrm). This method includes the steps of (a) contacting a test oocyte that expresses a functional ortholog of a Drosophila Matrimony polypeptide identified in a functional ortholog assay disclosed herein with a candidate compound and (b) determining whether the candidate compound causes a decrease in Mtrm function, an increase in Polo kinase function, nuclear envelop break down, and/or entry into prometaphase 1, wherein a candidate compound that decreases Mtrm function, increases Polo kinase function, triggers nuclear envelop break down (NEB) and/or entry into prometaphase 1 relative to a control cell that is not contacted with the candidate compound is indicative that the candidate compound may be effective to inhibit the ortholog of Drosophila Mtrm.

In the present invention, we are interested, inter alia, in elucidating the mechanisms that arrest meiotic progression at the end of prophase, but then allow onset of NEB and the initiation of meiotic spindle formation some 40 hours later. One intriguing possibility is that during this period of meiotic arrest the oocyte actively blocks the function of cell cycle regulatory proteins such as cyclin dependent kinase 1 (Cdk1), the phosphatase Cdc25 and Polo kinase (Polo), all of which promote meiotic progression, just as they do during mitotic growth. Recently, Polo was shown to be expressed in the germarium and required for the proper entry of Drosophila oocytes into meiotic prophase, as defined by the assembly of the SC [6]. Decreased levels of Polo resulted in delayed entry into meiotic prophase, while over-expression of Polo caused a dramatic increase in the number of cystocyte cells entering meiotic prophase, indicating that Polo is involved both in the initiation of SC formation and in the restriction of meiosis to the oocyte. How then is Polo, which is known to play multiple roles in promoting meiotic and mitotic progression [7,8], prevented from compelling the differentiated oocyte to proceed further into meiosis?

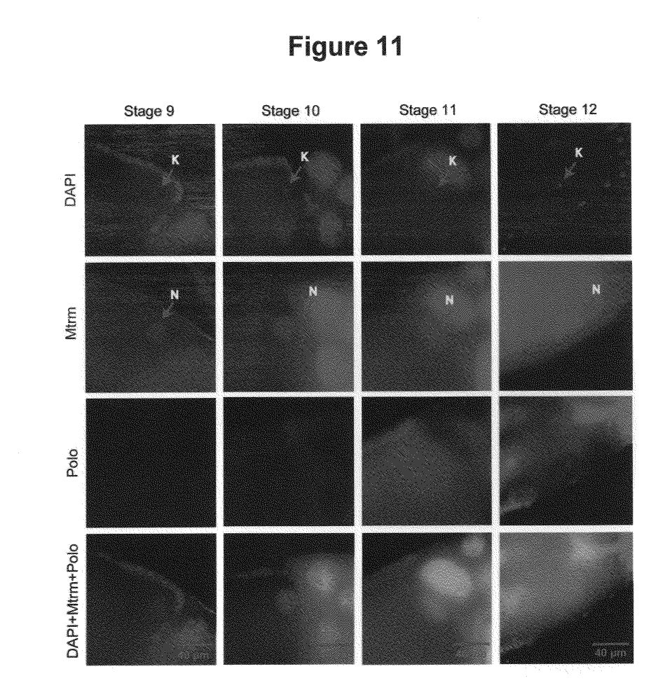

One component of this regulation may well lie in the fact that Polo is not expressed during much of oogenesis. As shown below, Polo is clearly visible in the germarium but is then absent until stage 11 when it begins to accumulate to high levels in the oocyte (see FIG. 11). We show here that a second component of Polo regulation is mediated by binding to the protein product of the matrimony (mtrm) gene (FBgn0010431), which occurs from stage 11 until the onset of NEB at stage 13. This binding serves to inhibit Polo in the early stages of its expression, and thus prevents precocious nuclear envelope breakdown.

The mtrm gene was first identified in a deficiency screen for loci that were required in two doses for faithful meiotic chromosome segregation [9]. mtrm/+ heterozygotes display a significant defect in achiasmate segregation (the meiotic process that ensures the segregation of those homologs that, for various reasons, fail to undergo crossingover). As a result of this defect, mtrm/+ heterozygotes exhibit high levels of achiasmate nondisjunction. As homozygotes, mtrm mutants are fully viable but exhibit complete female sterility. We show here that the Mtrm protein prevents precocious NEB. Indeed, as discussed below, the effects of reducing the dose of mtrm on meiotic progression and on chromosome segregation are easily explained as the consequence of precocious NEB at stages 11 or 12, and can be suppressed by simultaneously reducing the copy number of polo⁺. In addition, the effects of heterozygosity for loss-of-function alleles of mtrm can be phenocopied by increasing the copy number of polo⁺. These genetic interactions suggest that Mtrm negatively regulates Polo in vivo.

Interestingly, Mtrm was shown to interact physically with Polo by a global yeast two-hybrid study [10]. We demonstrate that this yeast two-hybrid finding reflects a true physical interaction in vivo by both co-immunoprecipitation studies and by Multidimensional Protein Identification Technology (MudPIT) mass spectrometry experiments which indicate that Mtrm binds to Polo with an approximate stoichiometry of 1:1. Moreover, ablating one of the two putative Polo binding sites on Mtrm by mutation prevents the physical interaction between Polo and Mtrm and renders the mutated Mtrm protein functionless. This experiment, along with genetic interaction studies, provides compelling evidence that the function of the binding of Mtrm to Polo is to inhibit Polo, and not vice versa.

The analysis of mtrm mutants allows us to examine the effects of premature Polo function during oogenesis. Our evidence shows that in the absence of Mtrm, newly synthesized Polo is capable of inducing NEB from stage 11 onward. As a result of this precocious NEB, chromosomes are not properly compacted into a mature karyosome and they are released prematurely onto the meiotic spindle. In many cases, the centromeres of achiasmate bivalents subsequently fail to co-orient.

### The Mtrm Gene Encodes a 217 Amino Acid Protein Whose Expression is Limited to the Period Between the End of Pachytene and the Onset of NEB

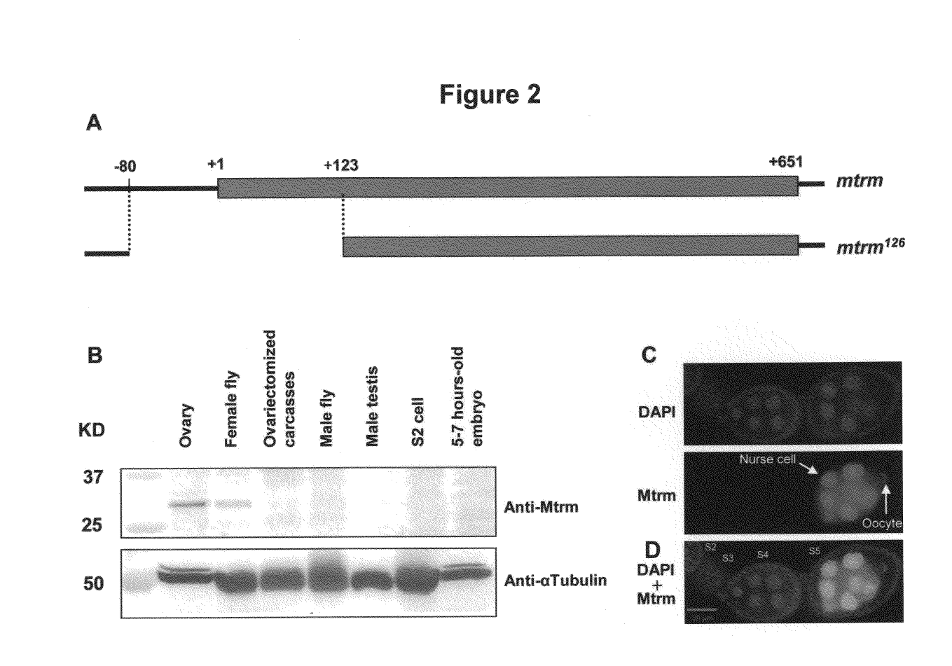

The mtrm gene was first identified as a dosage-sensitive meiotic locus. Heterozygosity for a loss-of-function allele of mtrm specifically induced the failed segregation of achiasmate homologs [9]. The mtrm gene encodes a 217 amino acid protein with two Polo Box Domain binding sites (STP and SSP) and a C-terminal SAM/Pointed domain (see, e.g., SEQ ID NO:13). The studies reported herein rely primarily on a null allele of mtrm (mtrm¹²⁶) that removes 80 bp of upstream sequence and the sequences encoding the first 41 amino acids of the Mtrm protein (see FIG. 2A).

Western blot analysis using an anti-Mtrm antibody reveals that Mtrm can only be detected in ovaries (FIG. 2B). This is consistent with a previous report by Arbeitman et al. [11] which showed that the expression profile of the mtrm gene product was strictly maternal, and that its expression was reduced greater than 10 fold over 0 to 6.5 hours of embryonic development. The specificity of this antibody is demonstrated by the fact that no signal was detected by either western blotting or by immunofluorescence of ovarioles homozygous for the mtrm¹²⁶ mutant (FIG. 2C). Immunofluorescence studies using the same antibody reveal that Mtrm is expressed as a diffuse nuclear protein in the oocytes and nurse cells beginning at stage 4-5 (see FIGS. 2C and 2D). As shown in FIG. 2C, the Mtrm signal was not restricted to the karyosome itself; but rather Mtrm seems to fill the space in the entire nucleus. Although Mtrm is restricted to the nucleus until approximately stage 10, it localizes throughout the oocyte in later stages. Mtrm brightly stains both the oocyte nucleus and cytoplasm between stage 11 and stage 12, but staining is greatly reduced at stage 13, the stage at which NEB occurs (FIG. 11).

### Reducing the Dosage of the Polo⁺ Gene Suppresses the Chromosome Segregation Defects Observed in Mtrm/+ Heterozygotes

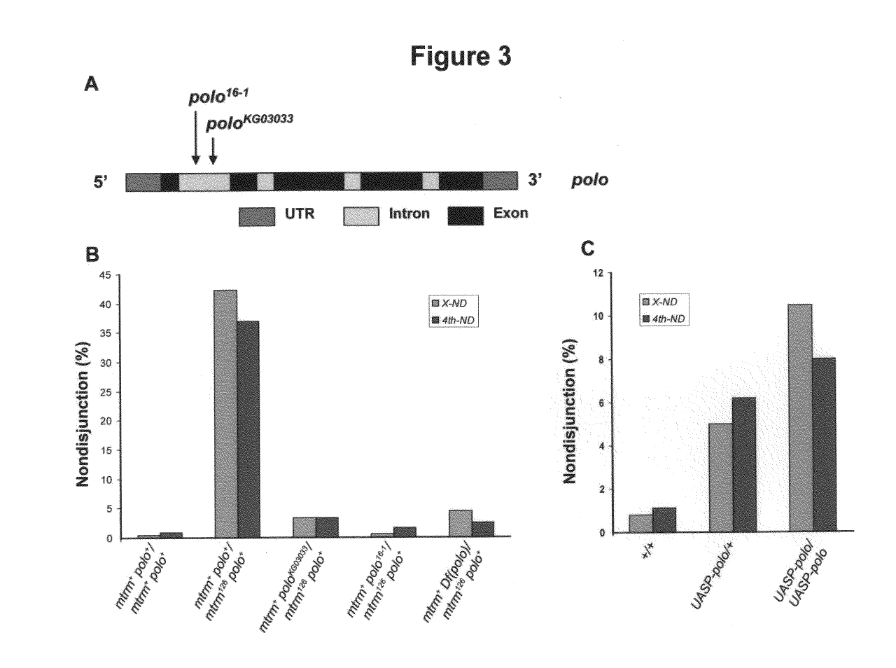

Mtrm/+ heterozygotes display a significant defect in the processes that ensure the segregation of achiasmate homologs. These meiotic defects are strongly suppressed by simultaneous heterozygosity for strong loss-of-function alleles of polo (FBgn0003124). The impetus for searching for a genetic interaction between mtrm and polo came from the finding that the mutants in the mei-S332 gene were partially suppressed by polo mutants [12]. Meiotic mis-segregation was measured by assaying X and 4^{th} chromosomal nondisjunction in females of the genotype FM7/X where FM7 is a balancer chromosome that fully suppresses X chromosomal exchange. The 4^{th} chromosome is obligately achiasmate. As shown in FIG. 3B, FM7/X; mtrm/+ females typically show frequencies of X and 4^{th} chromosome nondisjunction in the range of 35-45%, more than 100-fold above control values.

However, FM7/X; mtrm¹²⁶/+ females that were simultaneously heterozygous for either a deficiency (Df(3L)rdgC-co2) that uncovers polo or for either of two strong alleles of polo, polo^{KG03033} and polo¹⁶⁻¹ (see FIG. 3A), displayed greatly reduced levels of meiotic nondisjunction (see FIG. 3B). The fact that the polo^{KG03033} mutation is due to a P element insertion allowed us to demonstrate that the observed interaction with mtrm was indeed a direct consequence of a reduction in polo activity. Two precise excisions of this insertion were generated and neither was able to suppress the nondisjunctional effects observed in mtrm/+ heterozygotes (data not shown). In addition, we also demonstrated that the polo^{KG03033} allele was able to suppress the meiotic defects generated by heterozygosity for mtrm^{exc13}, an independently isolated allele of mtrm (data not shown).

Heterozygosity for these same loss-of-function alleles of polo has no detectable effect on meiotic chromosome segregation in mtrm⁺/mtrm⁺ females. In females of the genotypes FM7/X; polo^{KG03033}/+ or FM7/X; polo¹⁶⁻¹/+, the observed levels of nondisjunction for the X chromosome were 0.2% and 0.4%, respectively. Similarly, the observed levels of nondisjunction for the 4^{th} chromosome were 0.6% and 0.5%, respectively (n=1109 for FM7/X; polo^{KG03033}/+ and n=1226 for FM7/X; polo¹⁶⁻¹/+ females). These data alone are consistent with either a hypothesis in which Mtrm acts to inhibit Polo, and excess Polo creates a meiotic defect or a scenario in which Polo inhibits Mtrm, and the absence of sufficient Mtrm creates the defect. However, as we will show below, our additional data support the model whereby Mtrm inhibits Polo.

### Increasing the Dosage of Polo⁺ Partially Mimics the Effects of Mtrm and Enhances The Defects Observed In Mtrm/+ Heterozygotes

If reducing the quantity of Polo suppresses the meiotic defects observed in mtrm/+ females, then over-expression of Polo alone should mimic the effects of reducing the dosage of mtrm⁺ (i.e., we should see a chromosome segregation defect solely in the presence of increased dosage of polo⁺, even in mtrm⁺/mtrm⁺ oocytes). To test this hypothesis, we analyzed FM7/X females carrying two doses of a UASP-polo⁺ transgene construct driven by the nanos-GAL4 driver. As shown in FIG. 3C, expression of the UASP-polo⁺ transgene construct results in a dosage-dependent increase in the frequency of achiasmate nondisjunction for both the X and the 4^{th} chromosomes. Similar observations were made using chromosomal duplications that carry two copies of polo⁺ (Adelaide Carpenter, personal communication). Moreover, increasing the dose of Polo in females heterozygous for mtrm¹²⁶ resulted in severe meiotic defects. Females carrying a single copy of the UASP-polo⁺ transgene and which were also heterozygous for mtrm¹²⁶ were virtually sterile (data not shown). Thus, increasing the dosage of Polo enhances the defect observed in mtrm/+ heterozygotes by inducing sterility.

The genetic interaction between Mtrm and Polo during oogenesis is paralleled by their patterns of expression. Mtrm reaches its maximum level of expression from the end of stage 10 onward, filling the oocyte during stages 11-12, and then diminishes at stage 13. Analysis of Polo expression using an anti-Polo antibody [13,14] and wild-type oocytes revealed that Polo is present in the oocyte at low levels (except in the germarium) until stages 11 or 12 and then rapidly fills the oocyte cytoplasm from stages 12-13 onward (FIG. 11). Taken together, these data support a model in which the presence of Mtrm inhibits Polo in the early stages of expression, while permitting the function of Polo at stage 13, when Mtrm is degraded. Data directly demonstrating that assertion are provided below.

### Mtrm and Polo Physically Interact In Vivo

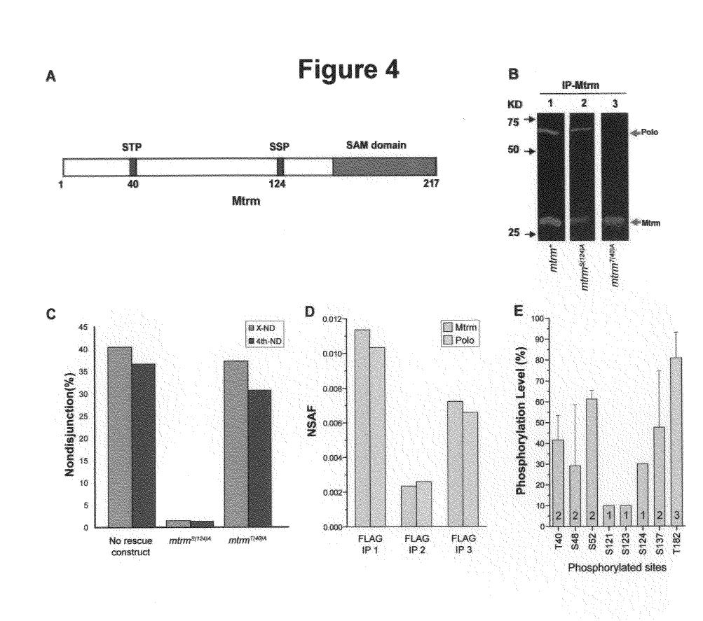

A large scale yeast two-hybrid screen identified Mtrm as a candidate interactor with Polo [10] and showed that Mtrm carries two putative PBD binding sites, STP and SSP (FIG. 4A). In order to confirm that Mtrm interacts with Polo physically in vivo, we performed co-immunoprecipitation experiments on wild-type ovary extracts using a polyclonal anti-Mtrm antibody. As shown in lane 1 of FIG. 4B, the anti-Mtrm antibody also precipitated Polo.

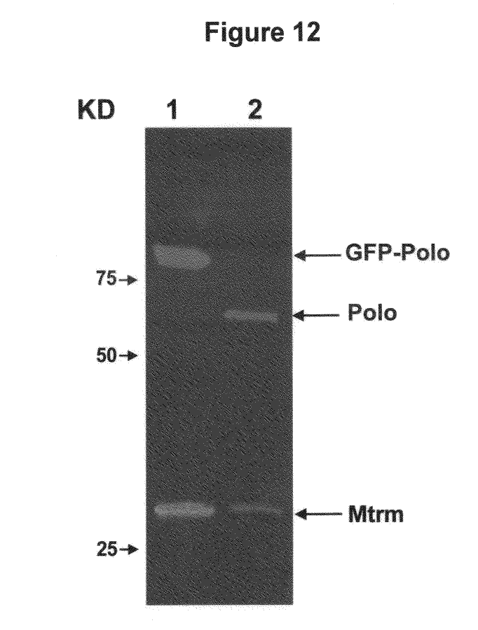

We used two separate approaches to confirm the interaction between Polo and Mtrm. In the first experiment, we used ovary extracts from females expressing a GFP-polo transgene [13] and performed the co-immunoprecipitation using an anti-GFP antibody. In the second experiment, we used ovary extracts from wild-type females and performed the co-immunoprecipitation using a monoclonal anti-Polo antibody [14]. In both experiments, we were able to show that Mtrm co-immuno-precipitated with Polo (FIG. 12).

In addition, MudPIT mass spectrometry reveals that Mtrm and Polo interact in oocytes with a stoichiometry of approximately 1:1. We analyzed three independent affinity purifications from ovarian extracts expressing a C-terminally 3×FLAG-tagged Mtrm and used MudPIT mass spectrometry [15] to identify interacting proteins. We then compared the identified proteins to those detected in five control FLAG immuno-precipitations from control (w¹¹¹⁸) flies. Among the proteins that showed reproducible and significant p values (p<0.001) identified in all three analyses, Polo was detected by multiple peptides and stands out as the only protein recovered at levels similar to those of Mtrm, as estimated by normalized spectral counts (NSAF) [16,17]. Although the NSAF values for Mtrm and Polo vary across the three biological replicates analyzed (FIG. 4C), the ratio between the two proteins remains constant with an average of 0.96±0.11, suggesting one Mtrm molecule binds to one molecule of Polo.

Thus, three lines of evidence demonstrate that Mtrm physically interacts with Polo: the yeast two-hybrid work [10]; our co-immunoprecipitation studies; and our MudPIT mass spectrometry experiments presented in this section. The observation of strong genetic interactions between mutants in these two genes (see FIG. 3) demonstrates a functional significance to this interaction.

Mutation of the First PBD Binding Site of Mtrm Both Prevents its Ability to Interact with Polo and Ablates Mtrm Function

Polo interacts with target proteins via the interaction of its Polo-box Domain (PBD) and the sequences STP or SSP on the target protein. In both of these PBD binding sites the center residues (threonine or serine) are phosphorylated to facilitate Polo binding [18-20]. Mtrm carries two potential PBD binding sites: STP with the central threonine at residue 40 and SSP with the central serine at residue 124 (FIG. 4A). To determine whether or not the interaction between Mtrm and Polo is mediated through the interaction of the Polo PBD with either or both of these two potential PBD binding sites, we created UASP-driven transgenes that carried mutations in either or both of the STP or SSP motifs. In each case, we mutated the central residue of the PBD binding sites on Mtrm to the non-phosphorylateable residue alanine. These mutants are denoted as mtrm^{T(40)A} which disrupts the STP motif and mtrm^{S(124)A} which disrupts the SSP motif. Each of these mutant constructs was expressed under the control of the nanos-GAL4 driver in a mtrm null background to insure that they were the only source of Mtrm protein in the oocytes. Co-immunoprecipitation experiments using anti-Mtrm antibodies revealed that Mtrm^{S(124)A} protein still interacted with Polo (FIG. 4B). However, Mtrm^{T(40)A} failed to bind to Polo (FIG. 4B), indicating that the STP residues define a motif critical for the Mtrm-Polo interaction. Mutation of both PBD sites also resulted in a version of Mtrm that did not interact with Polo (data not shown).

Because the interaction of Polo with target proteins via its Polo-box Domain (PBD) requires the phosphorylation of the center residues (threonine or serine) of the STP or SSP motifs [18-20], we searched the MS/MS dataset for phosphorylated peptides derived from Mtrm or Polo. For each of the detected sites, we estimated the levels of modification by dividing the number of spectra matching a particular phosphopeptide by the total spectral count for this peptide (FIG. 4D). We were able to detect phosphorylation on both T40 and S124, although, in agreement with the second PBD not being the primary binding site, S124 phosphorylation was found less reproducibly (FIG. 4E). In addition, Mtrm S48, S52 and S137 were found phosphorylated at reproducibly high levels in two out of three experiments. We also observed that Polo T182 was detected as phosphorylated at high levels (over 80%) in all three immunoprecipitations, indicating that those Polo proteins that are bound to Mtrm were fully activated [21].

Not only is the STP motif important for Polo binding, but it is also required for proper Mtrm function (FIG. 4E). We assayed the frequency of nondisjunction in females expressing either the mtrm^{S(124)A} or the mtrm^{T(40)A} construct in the germ lines of FM7/X; mtrm/+ heterozygotes (FIG. 4C). Although the mtrm^{S(124)A} construct was able to rescue the meiotic defects seen in mtrm/+ heterozygotes, the mtrm^{T(40)A} construct failed to rescue the mtrm defect, and maintained the high nondisjunction frequency seen in FM7/X; mtrm/+ heterozygotes. A similar failure to rescue was observed using a double mutant construct that carried both the mtrm^{S(124)A} and the mtrm^{T(40)A} mutations (data not shown). Based on these observations, we conclude that the STP site is critical for Mtrm function and the T(40)A mutation ablates Mtrm function as a direct consequence of a failure to interact with Polo.

### Mtrm Functions as an Inhibitor of Polo

In the previous sections, we have presented three separate lines of evidence that Mtrm acts to inhibit Polo function and not vice versa. First, effects of heterozygosity for mtrm can be suppressed by a corresponding reduction in the dose of polo⁺. Second, we observed that the phenotype created by reducing the dose of mtrm⁺ can be mimicked by increasing the dose of Polo. Third, and most importantly, the observation that mutating the STP Polo binding site by a conservative amino acid replacement (STP->SAP) ablates Mtrm function argues strongly that Mtrm functions as an inhibitor of Polo. Were it the case that Polo inhibits Mtrm, one would expect loss of the Polo interacting site to produce a hyper-functional Mtrm, not a non-functional protein.

### As Either a Heterozygote or a Homozygote, Mtrm Causes Precocious Nuclear Envelope Breakdown

The early stages of meiosis appear normal in both mtrm/+ and mtrm/mtrm oocytes. The germarium and early stages appear morphologically normal and at least in mtrm/+ oocytes both recombination and SC assembly are indistinguishable from normal ([9] and unpublished data). However, following stage 11, the period during which Mtrm is maximally expressed, we observed multiple defects in oocyte maturation in both mtrm/+ and mtrm/mtrm oocytes. Most critically, we show that a loss-of-function allele of mtrm induces precocious NEB in a dosage-dependent manner.

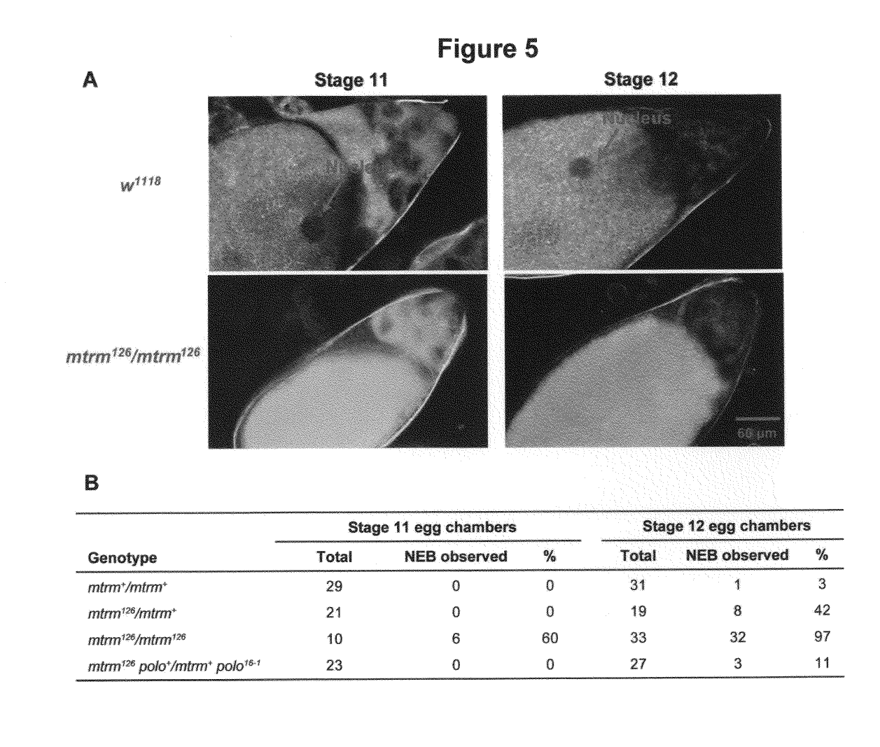

In wild-type oocytes, NEB usually does not occur until stage 13; only a single case of NEB at stage 12 was observed among the 61 stage 11 and 12 wild-type oocytes examined (see FIG. 5). However, in mtrm¹²⁶/+ heterozygotes more than a third of stage 12 egg chambers exhibited NEB. To ensure that the precocious NEB defect is the consequence of reducing the copy number of mtrm⁺, we repeated these experiments using females heterozygous for an independently isolated allele of mtrm, mtrm^{exc13}. These females also displayed precocious NEB at stage 12 (data not shown). As is the case for the chromosome segregation defects observed in mtrm/+ oocytes, the precocious NEB that is seen in mtrm¹²⁶/+ heterozygotes is strongly suppressed by simultaneous heterozygosity for a loss-of-function allele of polo (see FIG. 5B), suggesting that the timing of NEB is determined by the relative abundances of Mtrm and Polo. This conclusion is further strengthened by the observation that over-expression of Polo (using a UASp-polo+ transgene driven by the nanos-GAL4 driver) increases the frequency of precocious NEB in mtrm¹²⁶/+heterozygotes by nearly two-fold (from 42% to 77%). The extent of the precocious NEB defect is even more evident in mtrm¹²⁶ homozygotes. As shown in FIG. 5, NEB had already occurred in 32 out of 33 stage 12 oocytes examined and in 6 of 10 stage 11 oocytes examined. Thus, the loss of Mtrm causes precocious NEB in a dosage-dependent fashion. Taken together, these data argue that the presence of Mtrm prevents Polo from inducing NEB until stage 13, and that a reduction or absence of available Mtrm allows the Polo synthesized during stages 11 and 12 to initiate NEB.

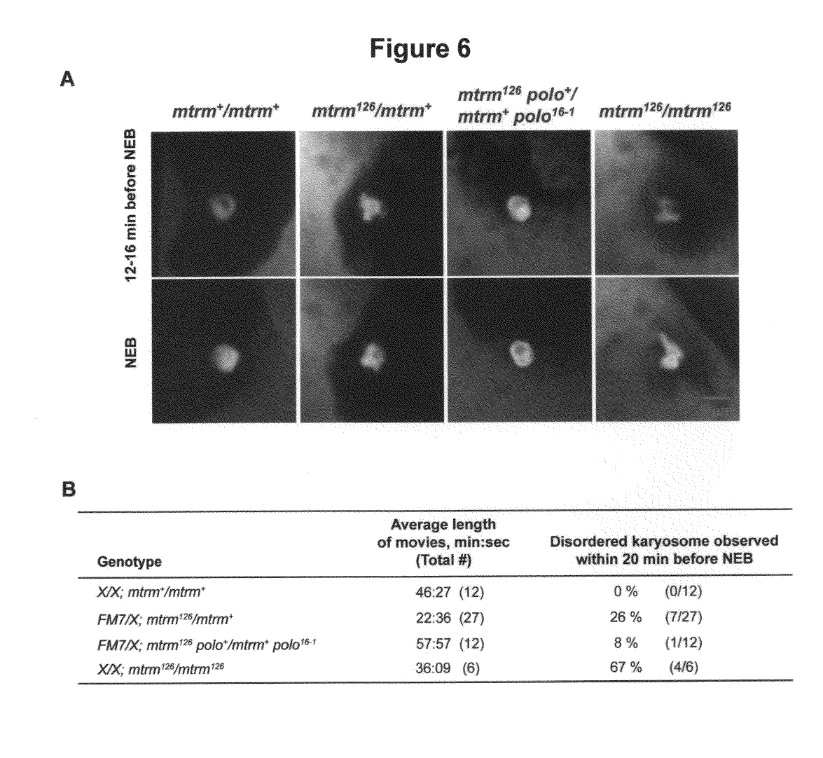

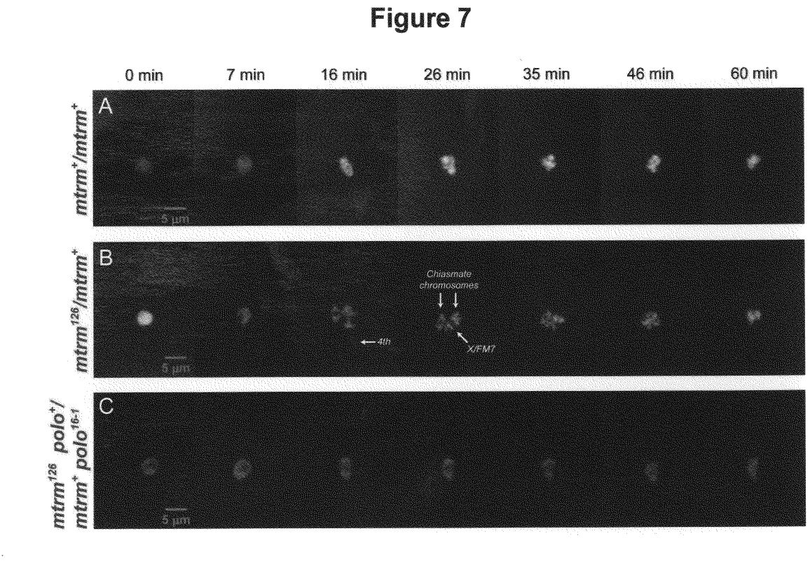

The precocious breakdown of the nuclear envelope at stages 11 to 12 is significant because the karyosome undergoes dramatic changes in structure during this period [2]. As noted above, in stages 9-10, the karyosome expands to the point that individual chromosomes can be detected [22-24]. These chromosomes re-condense into a compact karyosome during stages 11 to 12, the exact time at which a reduction in the level of Mtrm causes precocious NEB. Thus, the early NEB events promoted by heterozygosity for mtrm might be expected to result in the release of incompletely condensed or disordered karyosomes. To test this hypothesis, we examined karyosome morphology during the 20 minutes that preceded NEB in wild-type, mtrm¹²⁶/mtrm⁺, and mtrm¹²⁶ polo⁺/mtrm⁺ polo¹⁶⁻¹ oocytes. As shown in FIG. 6, in only 2 out of 28 (7%) wild-type oocytes with incompletely compacted or disordered karyosomes were observed. However, 7 out of 27 (26%) mtrm¹²⁶/mtrm⁺ oocytes displayed a disordered karyosome, an effect that was largely suppressed (to 8%) by simultaneous heterozygosity for polo¹⁶⁻¹ (FIG. 6). These data support the view that the precocious NEB induced by lowering the level of Mtrm results in the release of improperly formed karyosomes into the cytoplasm and are again consistent with the possibility that Mtrm inhibits meiotic progression through its effects on Polo.

Mtrm is Also Required to Maintain Karyosome Structure after NEB

The karyosome plays a critical role in directing the formation of the acentriolar spindle in Drosophila oocytes. In 8 out of 9 (89%) wild-type oocytes, the karyosome remains associated even after NEB; it is then surrounded by microtubules and forms a bipolar meiotic spindle (FIG. 7). At metaphase I, chiasmate chromosomes are still condensed into a single mass at the metaphase plate in a tapered bipolar spindle [25-28].

However, in FM7/X; mtrm¹²⁶/mtrm⁺ oocytes the karyosome usually dissolved within 10-20 minutes following NEB and the individual bivalents became clearly visible (FIG. 7). In 15 out of 17 (88%) FM7/X, mtrm¹²⁶/mtrm⁺ oocytes examined, the chromosomes were individualized during spindle assembly. Indeed, in 14 of these movies all three pairs of major chromosomes were physically separated at some point during the time course of imaging (in the remaining case, the three bivalents could be distinguished but were still physically associated). As disclosed previously herein, despite this dissociation into individual bivalents, in most oocytes the chromosomes are capable of re-aggregating into a single mass and eventually forming a bipolar spindle.

A striking example where all four chromosome pairs can be clearly distinguished is the image taken 26 minutes after NEB for FM7/X; mtrm¹²⁶/mtrm⁺ oocytes (FIG. 7). In those oocytes in which bivalent individualization was observed, the two major autosomes appeared to be held together by at least two chiasmata (one on each arm), suggesting that sister-chromatid cohesion along the euchromatic arms of these chromosomes still persists. The two X chromosomes remain physically associated, despite the lack of chiasmata, presumably as a consequence of the maintenance of heterochromatic pairing [29,30].

Since the nondisjunction of achiasmate chromosomes observed in mtrm¹²⁶/mtrm⁺ heterozygotes was suppressed by heterozygosity for loss-of-function alleles of polo, we next tested whether a polo mutation could also suppress this karyosome maintenance defect. As shown in FIG. 7, bivalent individualization was only observed in 3 out of 13 (23%) of FM7/X; mtrm¹²⁶ polo⁺/mtrm⁺ polo¹⁶⁻¹ oocytes, and thus 77% of the oocytes maintained the karyosome as a single mass throughout the process of spindle assembly. These data are consistent with the genetic data presented above: reducing the dose of polo⁺ strongly suppresses the deleterious effects of heterozygosity for mtrm.

### The Defects in Karyosome Maintenance are Followed by Defective Co-Orientation of Achiasmate Centromeres on the Meiotic Spindle

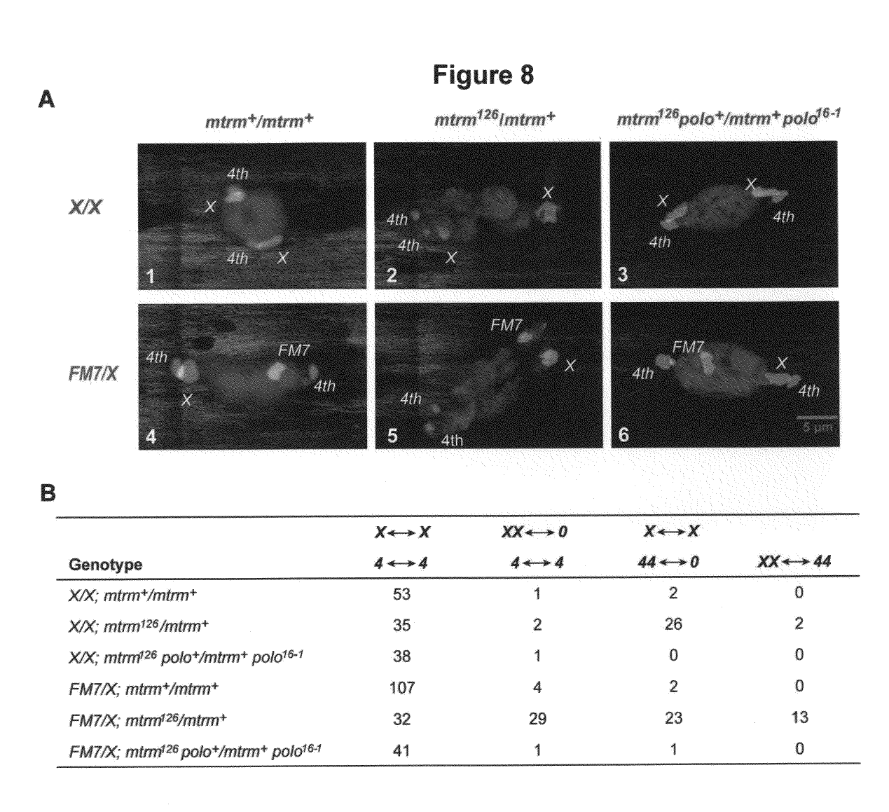

Because the karyosomes of mtrm/+ females were poorly formed prior to NEB and usually transiently dissolved to individual bivalents shortly after NEB (see above), we also examined centromere co-orientation on bipolar prometaphase spindles using FISH probes (see Examples) directed against the X and 4^{th} chromosomes (FIG. 8) in both wild-type and mtrm/+ oocytes.

In wild-type oocytes, the vast majority of most X and 4^{th} chromosome centromeres co-oriented properly (see FIG. 8). The frequencies of abnormal centromere co-orientation in oocytes with chiasmate X chromosomes (XX) were only 2% for the X chromosome and 4% for the 4^{th} chromosome. In FM7/X females, where X chromosomal crossingover is blocked, the frequencies of abnormal co-orientation were still quite low (4% for the X chromosome and 2% for the 4^{th}) However, co-orientation of achiasmate centromeres was often aberrant in mtrm/+ heterozygotes, such that the centromeres of both homologs were often oriented toward the same pole (FIG. 8A). In these cases, the two homologs also occupied different arcs of the meiotic spindle, a feature that is rarely, if ever, observed in wild-type oocytes. In chiasmate X females, 43% of observed oocyte nuclei displayed an aberrant co-orientation of 4^{th} chromosome centromeres, and 6% of these oocytes displayed aberrant X centromere co-orientations (FIG. 8B); these oocytes likely reflect the 8-10% of oocytes that fail to undergo crossingover even in females bearing structurally normal X chromosomes. The defect in 4^{th} chromosome centromere co-orientation was fully suppressed by simultaneous heterozygosity for polo¹⁶⁻¹ (FIGS. 8A and 8B).

As expected, due to the suppression of X chromosomal crossingover in FM7/X females, mtrm/+ heterozygotes displayed frequent abnormal centromere co-orientation for both X and 4^{th} chromosomes, i.e. 43% for X chromosomes and 37% for 4^{th} chromosomes (FIG. 8B). These results indicate that the mtrm heterozygotes display an obvious defect in centromere co-orientation. However, once again, both the defect in X and 4^{th} chromosome centromere co-orientation was fully suppressed by simultaneous heterozygosity for polo¹⁶⁻¹. Thus, as was the case with the previously considered defects, the deleterious effects of reducing the amount of available Mtrm can be suppressed by a simultaneous reduction in the amount of Polo.

The data presented above argue that Mtrm serves to inactivate newly-synthesized Polo during the period of meiotic progression that precedes NEB. An excess of functional (un-bound) Polo, produced by reducing the amount of available Mtrm, causes the early onset of NEB. This early entry into prometaphase releases an immature karyosome into the cytoplasm, which then fails to properly align the centromeres of achiasmate chromosomes on the prometaphase spindle. These observations raise a number of questions ranging from the role of Polo in mediating the G2/M transition in oogenesis to the role of the karyosome structure in facilitating the proper segregation of achiasmate chromosomes.

### Polo Plays a Critical Role in Initiating the G2/M Transition in Oogenesis by Regulating Cdc25

The trigger for the G2/M transition is activation of Cdk1 by Cdc25 (reviewed by [31]), and multiple lines of evidence suggest that Polo can activate Cdc25 [32]. First, in C. elegans, RNAi experiments demonstrate that ablation of Polo prevents NEB [33]. Second, the Xenopus Polo homolog Plx1 is activated in vivo during oocyte maturation with the same kinetics as Cdc25. Additionally, microinjection of Plx1 accelerates the activation of both Cdc25 and cyclinB-Cdk1 [34]. Moreover, microinjection of either an anti-Plx1 antibody or kinase-dead mutant of Plx1 inhibited the activation of Cdc25 and its target cyclinB-Cdk1. A later study by Qian et al. demonstrated that injection of a constitutively active form of Plx1 accelerated Cdc25 activation [35]. As pointed out by these authors, these studies support “the concept that Plx1 is the ‘trigger’ kinase for the activation of Cdc25 during the G2/M transition.” Finally, a small molecule inhibitor of Polo kinase (BI 2536) also results in extension of prophase [36]. These data are consistent with the view that the presence of functional (un-bound) Polo plays a critical role in ending the extended G2 that is characteristic of oogenesis in most animals. We should note by Riparbelli et al. [37] that the careful study of female meiosis in polo¹ homozygotes failed to observe a defect in the timing of NEB. However, as disclosed previously herein, polo¹, a missense mutant that is viable even over some deficiencies and does not suppress mtrm, is the weakest of the known polo mutants and it is thus reasonable that no defect was observed.

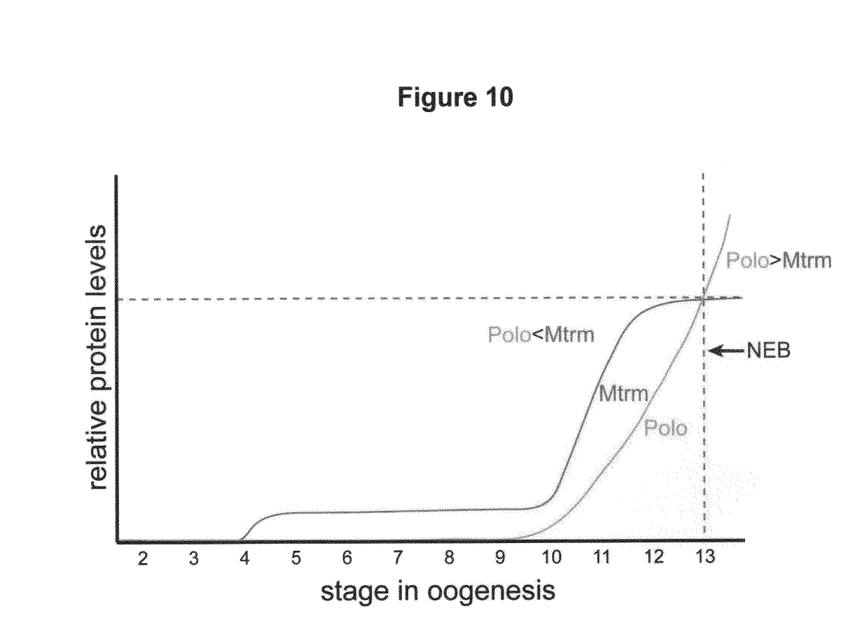

In light of these data, it is tempting to suggest that in wild-type Drosophila oocytes the large quantity of Mtrm deposited into the oocyte from stage 10 onward inhibits the Polo that is either newly synthesized or transported into the oocyte during stages 11 to 12. However, at stage 13 an excess of functional Polo is created when the number of Polo proteins exceeds the available amount of inhibitory Mtrm proteins. This unencumbered, and thus functional Polo then serves to activate Cdc25, initiating the chain of events that leads to NEB and the initiation of prometaphase. In the absence of a sufficient amount of Mtrm, an excess of Polo causes the precocious activation of Cdc25, and thus an early G2/M transition. A model describing this hypothesis is presented in FIG. 10. Based on this model, one can visualize that decreasing the dose of Mtrm or increasing the dosage of Polo will hasten NEB, while simultaneous reduction in the dosage of both proteins should allow for proper timing of NEB.

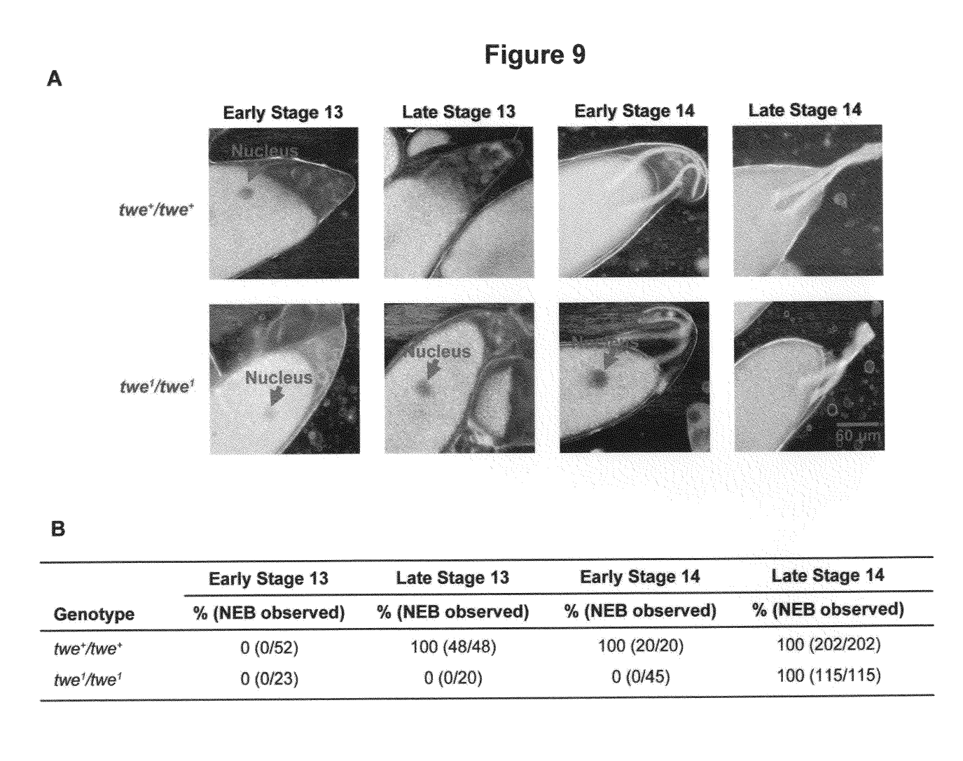

Two lines of evidence directly support a model in which Mtrm exerts its effect on Polo, with respect to preventing precocious NEB, by blocking the ability of Polo to activate Cdc25. First, as shown in FIG. 9, mutants in the Drosophila cdc25 homolog twine (FBgn0002673) fail to undergo NEB in stage 13. In addition, heterozygosity for twine also decreases the frequency of precocious NEB in mtrm¹²⁶/+ heterozygotes from 42% (see FIG. 5) to less than 10% (7/72).

### Mtrm Inhibition of Polo

Mtrm's first PBD binding site (T40) is required for its interaction with Polo. Mtrm T40 has to be first phosphorylated by a priming kinase, such as one of the Cdks or MAPKs, and was indeed detected as phosphorylated in the mass spectrometry dataset. The NetPhosK algorithm [38] predicts T40 to be a Cdk5 site, and the serines immediately distal to T40, S48 and S52, which were also detected as phosphorylated (FIG. 4E), are sites for proline-directed kinases such as Cdk or MAPK sites as well. The other prominent phosphorylation event occurs at S137, which could be a Polo phosphorylation site because it falls within a Polo consensus (D/E-X—S/T-Ø-X-D/E). Although the combined sequence coverage for Mtrm was 44%, indicating that some phosphorylated sites might have been missed, Mtrm S137 is a suitable binding site for activated Polo, in agreement with the processive phosphorylation model [18]. At this point of our studies, Mtrm T40 priming kinase or the kinase responsible for Polo activating phosphorylation on T182 has not been identified.

The finding that Polo not only is able to bind to Mtrm in vivo in a 1:1 ratio, but also is fully phosphorylated on T182 in its activation loop [21] suggests a method by which Mtrm serves to inhibit Polo. In general, enzymes are usually not recovered from affinity purifications at levels similar to their targets. They do not form stable complexes, but rather transient interactions with their substrates, which is how efficient catalysis is achieved. Here, Mtrm is able to sequester activated Polo away in a stable binary complex over a long period of time. It is only when this equilibrium is disturbed at the onset of stage 13 by the production of an excess of Polo (as suggested in FIG. 10) or by degradation of Mtrm that Polo can be released. The molecular determinants of the Mtrm::Polo sequestration event are not clear, but it would be interesting to test whether the serines found phosphorylated in the vicinity of Mtrm PBD binding sites play a role in locking the binary complex into place.

### Mtrm Exerts its Effects on Achiasmate Nondisjunction Via a Cdc25-Independent Pathway

Our data demonstrate that a reduction in the levels of Mtrm results in the release of an incompletely compacted karyosome that rapidly dissolves into individual bivalents during the early stages of spindle formation. For chiasmate bivalents this is apparently not a problem because they still co-orient correctly (for example, the chiasmate X chromosomes shown in FIG. 8 still achieve proper co-orientation in the vast majority of oocytes). However, the nonexchange bivalents frequently fail to co-orient properly such that both homologs are oriented toward the same pole (but often occupy two different arcs of the spindle). This initial failure of proper co-orientation leads to high frequencies of nondisjunction as demonstrated by the genetic studies and analysis of metaphase I images presented in Harris et al. (2003) [9].

Although achiasmate homologs are properly co-oriented in wild-type oocytes [29,30], we have noted previously such homologs can often vacillate between the poles such that two achiasmate homologs are often found on the same arc of the same half-spindle during mid- to late prometaphase ([25] and unpublished data). These chromosomes are often observed to be physically associated. This situation is quite different from the defect observed in mtrm/+ heterozygotes where the homologs are neither physically associated nor on the same arc of the spindle.

It is tempting to suggest that the chromosome segregation defects we observe in mtrm/+ heterozygotes are simply the result of precocious release of an incompletely re-compacted karyosome. According to this explanation, the defects observed in meiotic chromosome segregation are solely the consequence of premature NEB. (Implicit in this model is the assumption that it is the events that occur during karyosome re-compaction, at stages 11 and 12, that serve to initially bi-orient achiasmate chromosomes and we do not have direct evidence to support such a hypothesis.)

Alternatively, Polo plays multiple roles in the meiotic process [7,8], and it is possible that the chromosome segregation defects we see represent effects of excess Polo that are un-related to the precocious breakdown of the nuclear envelope. Such a view is supported by two observations. First as shown in FIG. 7, the bivalent individualization observed after NEB in mtrm/+ oocytes does not disrupt FM7-X pairings. Second, although heterozygosity for twine in mtrm¹²⁶/+ heterozygotes suppresses the frequency of precocious NEB from 42% (see FIG. 5) to less than 10% (7/72), but two alleles of twine tested (twe¹ and twe^{k08310}) failed to suppress the levels of meiotic nondisjunction observed in FM7/X; mtrm¹²⁶/+ heterozygotes. These data suggest that the effects of excess Polo on nondisjunction may not be regulated via Cdc25/Twine, but rather by the effects of excess Polo on some other, as yet unidentified Polo target. This suggests that the effects of Mtrm on the level of Polo might affect multiple Polo-related processes.

Support for such an idea that Mtrm can inhibit Polo-regulated proteins that are un-related to NEB comes from the observation that the ectopic expression of Drosophila Mtrm in S. pombe blocks karyokinesis, producing long multi-septate cells with only one or two large nuclei ([39], Bruce Edgar, personal communication). This phenotype is similar, if not identical to that, exhibited by mutants in the S. pombe Polo homolog plo1 (Plo1, CAB11167), which fail in later stages of mitosis due to the role of Plo1 in activating the septation initiation network to trigger cytokinesis and cell division. However, Plo1 also plays a role in bipolar spindle assembly that might also be inhibited in the Mtrm expressing cells, but this function of Plo1 is less well understood.

Thus, the possibility exists that the effect of mtrm mutants on meiotic chromosome segregation may well not be the direct consequence of early NEB, but rather due to the role of Polo in other meiotic activities, such as spindle formation or the combined effects of these defects with precocious NEB. Efforts to identify such processes and their components are underway in the lab.

Finally, we should note that while Mtrm is the first known protein that is able to inactivate Polo by physical interaction to Polo itself; there is certainly additional mechanisms of Polo regulation. For example, Archambault et al. [40] have described mutants in the gene, which encodes Greatwall/Scant kinase (FBgn0004461) that have both late meiotic and mitotic defects. Although there is no evidence for a physical interaction between these two kinases, the authors speculate that the function of the Greatwall kinase serves to antagonize that of Polo. The Scant mutations create a hyperactive form of Greatwall, which might be expected to lower the dosage of Polo, and thus perhaps partially suppress the defects observed in mtrm/+ heterozygotes. Indeed, exactly such a suppressive effect has been observed in Scant homozygotes (however, this suppression is much weaker than that obtained by heterozygosity for loss of function alleles of polo).

### SUMMARY

The data presented above demonstrate that Mtrm acts as a negative regulator of Polo during the later stages of G2 arrest during meiosis. Indeed, both the repression of Polo expression until stage 11 and the inactivation of newly synthesized Polo by Mtrm until stage 13 play critical roles in maintaining and properly terminating G2 arrest. Our data suggest a model in which the eventual activation of Cdc25 by an excess of Polo at stage 13 triggers NEB and entry into prometaphase. Although our data do shed some light on the mechanism by which Mtrm inhibits Polo, it is not entirely clear whether Polo's ability to phosphorylate targets other than Cdc25 might be blocked by Mtrm::Polo binding. These issues will clearly need to be addressed in future studies. Finally, we note that although small molecule inhibitors of Polo have been identified [36], Mtrm represents the first case of a protein inhibitor of Polo. It would be most exciting to identify functional orthologs of Mtrm outside of the genus Drosophila. Perhaps that might best be accomplished through a screen for oocyte-specific Polo-interacting proteins.

The invention now being generally described, it will be more readily understood by reference to the following examples, which are included merely for purposes of illustration of certain aspects and embodiments of the present invention, and are not intended to limit the invention.

### EXAMPLES

### Drosophila Stocks

Throughout this study a w¹¹¹⁸ stock served as our normal sequence X wild-type control, and for achiasmate X-chromosome studies, FM7/yw was used as wild-type control. The GFP-polo stock was kindly provided by Adelaide Carpenter. The nanos-GAL4 driver was used to express UASP-driven transgenes (see below) in the ovary. All polo mutants, the P element insertion mutant, and deficiencies related to mtrm were acquired from the Bloomington Drosophila Stock Center.

### Isolation and Characterization of a Null Allele of Mtrm

A P-element insertion mutant, KG08051, causing a mutation in the mtrm gene and exhibiting high levels of nondisjunction for achiasmate chromosomes [9] was obtained from the Bloomington Drosophila Stock Center. Although Harris et al. (2003) [9] positioned the insertion site for this transposon 90-bp upstream of the first ATG in the mtrm coding sequence, re-sequencing indicates that the true insertion site is in fact 80-bp upstream of the first ATG in the mtrm coding sequence (see, e.g., SEQ ID NO:12). mtrm¹²⁶ was generated by imprecise excision from the insertion of a null allele of mtrm. It is a deletion that removes 80-bp of 5′-UTR and 123-bp of coding sequence, deleting the first 41 amino acids (FIG. 2A). RT-PCR and Western blotting confirmed that mtrm¹²⁶ homozygotes had no transcripts and no protein expression (data not shown). Like the original P element insertion mutant, mtrm¹²⁶ showed a dosage-sensitive effect on meiotic nondisjunction that was specific to achiasmate chromosomes and homozygous sterile females (homozygous males are fully fertile and meiotic segregation is normal in both mtrm heterozygotes and homozygotes).

### Construction of Transgene Plasmids

To construct the UASp-polo⁺ transgene, we amplified a 1.74-kb XhoI-XbaI polo fragment from reverse transcribed cDNA by PCR using the primers 5′-ctcgaggatggccgcgaagcccgaggataag-3′ (SEQ ID NO: 1) and 5′-tctagattatgtgaacatcttctccagcattttcc-3′ (SEQ ID NO: 2). The polo fragment was cloned into the pBluescript to generate pBlue-polo-cDNA. Then, a polo fragment was obtained by digestion with KpnI and XbaI from pBlue-polo-cDNA and cloned into the pUASp vector [41] to produce pUASp-polo⁺. The UASp-polo⁺ cassette in this plasmid was sequenced for confirmation. The transformation of the pUASp-polo⁺ and other plasmids (see below), to generate transgenic flies, was conducted by Genetic Services, Inc. in Boston, Mass.

To place the 3×Flag downstream of mtrm, the PCR amplified 687-bp mtrm+1.5×-Flag fragment was created using primer pKpnI-mtrm-5,5′-ggggtaccaa atggagaattctcgcacgcccacgaacaag-3′ (SEQ ID NO: 3), and primer mtrm-3-flag(1.5×), 5′-gtccttgtagtccttgtcatcgtcgtccttgtagtcaagagtgtggagcacatccatgatacgg-3′ (SEQ ID NO: 4). Then the 687-bp mtrm+1.5×-Flag was amplified with the flag(3×)stop-XbaI primer, 5′-gctctagattacttgtcatcgtcgtccftgtagtccttgtcatcgtcgtccttgtagtccttgtcatcgtcgtccttg-3′ (SEQ ID NO: 5), to produce the KpnI-XbaI mtrm-Flag(3×) fragment. The fragment was then cloned into the pUASP vector [41] to produce pUASP-mtrm-flag(3×).

The Mtrm protein possesses two potential PBD binding sites: STP with the central threonine at residue 40 and SSP with the central serine at residue 124 (FIG. 4A). In order to mutate the central residues to alanine in each motif, PCR assembly was used to make two separate codon changes in the mtrm gene, one at +118 from ACT to GCT to produce mtrm^{T(40)A} and the other at +370 from CAG to CGC to produce mtrm^{S(124)A}. In order to mutate the STP motif, primer pmtrm-mut-ATG: 5′-cggggtaccaaaagatggagaattctcgcacgcccacgaacaagac-3′ (SEQ ID NO: 6) and primer pmtrm-STPre: 5′-gagaftgggcgaacggaagttgccaaagatcggagcagagcatcgcacgttggaggtgttcaccttcag-3′ (SEQ ID NO: 7) were used to amplify a 150-bp fragment for 5′-terminus of mtrm. The rest of mtrm was amplified with primers pmtrm-STP: 5′-ctgaaggtgaacacctccaacgtgcgatgctctgctccgatctttggcaacttccgttcgcccaatctc-3′ (SEQ ID NO: 8), and pmtrm-mut-TAA: 5′-gctctagattaaagagtg tggagcacatccatgatacgcttgc-3′ (SEQ ID NO: 9) to produce a 520-bp fragment. The 150-bp and 520-bp fragments were combined in equal amounts and amplified by PCR to assemble the full length KpnI-XbaI mtrm^{T(40)A} gene introducing a point mutation. The KpnI-XbaI mtrm^{T(40)A} was cloned in to pUASP to generate pUASP-mtrm^{T(40)A}. After confirmation by sequencing the plasmid was used for genetic transformation.

To construct the mtrm^{S(124)A} transgene, primer pmtrm-mut-ATG and primer pmtrm-SSPre: 5′-ggtctccatattcgagtcatccgaacaggtatccggggcgctgcagctct-3′ (SEQ ID NO: 10) were used to amplify a 420-bp fragment of the 5′-terminus of mtrm. The 3′-terminus of mtrm was amplified by using primer pmtrm-SSP: 5′-agagctgcagcgccccggatacc tgttcggatgactcgaatatggagacc-3′ (SEQ ID NO: 11) and primer pmtrm-mut-TAA to produce a 300-bp fragment. The two fragments in equal molar amounts were amplified by PCR to assemble a full length KpnI-XbaI mtnm^{S(124)A} gene with a point mutation introduced. The KpnI-XbaI mtrm^{S(124)A} was cloned in pUASP to generate pUASP-mtrm^{S(124)A}. The plasmid was used for genetic transformation after confirmation by sequencing.

### Antibodies

The mtrm gene was cloned into a pET-21a vector (Norvagen). 6×His-tagged Mtrm was expressed in the bacterial strain BL21 (DE3), isolated and purified using the Probed Purification System (Invitrogen) and used to raise rabbit and guinea pig polyclonal antisera by Cocalico Biologicals Inc in Reamstown, Pa. Affinity purification of the antiserum against Mtrm was performed by using a Sulfolink kit from the Pierce Company. Mouse monoclonal anti-Polo antibody was kindly provided by Moutinho-Santos [1,3]. Anti-GFP antibody from rabbits was purchased from Abcam Inc (Cambridge, Mass.).

### Immunostaining for Mtrm Localization

To prepare ovaries to fix for immunostaining, female fly preparation and ovary dissection were conducted as described in Xiang and Hawley (2006) [30]. Whole ovaries were collected and kept in 0.75 ml 1× Robb's solution during the dissection. After egg chambers were manually teased apart, the ovaries were transferred to an Eppendorf tube. Then, 0.25 ml 16% formaldehyde was added and incubated for 15 minutes. The ovaries were washed three times in PBS+0.1% Triton X-100 (PBST) for 10 minutes each. After washing three times in PBST, they were incubated in PBST with 5% goat serum for at least two hours at 4° C. with gentle shaking before being incubated overnight with primary antibodies. Egg chambers were washed four times in PBST and then incubated with proper fluorescently-labeled secondary antibodies for 4 hours at room temperature. Egg chambers were stained for ten minutes in PBST with 0.5 μg/ml DAPI and re-washed four times in the solution for a total of 40 minutes. The egg chambers were mounted on slides in Vectashield for analysis. Microscopy observation was conducted using a DeltaVision microscopy system (Applied Precision, Issaquah, Wash.) as described in Xiang and Hawley (2006) [30].

### Immunoprecipitations

To prepare the ovary extract for immunoprecipitation, ovaries from 100 yeast-fed female flies were dissected in 1×PBS. The ovaries were homogenized in an Eppendorf tube at 4° C. by a small pestle in 0.5 ml of ovary extract buffer containing 25 mM Hepes (pH 6.8), 50 mM KCl, 1 mM MgCl₂, 1 mM DTT and 125 mM sucrose with protease inhibitors cocktail (Calbiochem). The extract was centrifuged at 14000×g for 15 minutes at 4° C. and the supernatant was collected.

Protein A agarose beads were used for binding polyclonal antibodies from rabbit and guinea pig. Protein G agarose beads were used for binding monoclonal antibody from mouse. 50 ul of protein A or G-coated agarose was washed three times with PBST (PBS+0.1% Triton X-100). 10 ul of antibody was added to the beads in a final volume of 500 ul of PBS and mixed on a shaker for 1 hour at 4° C. The beads then were washed twice with PBST. The ovary extract was immunoprecipitated with the beads for 1 hour at 4° C. with continual shaking. After recovery by centrifugation at 1000×g for 3 minutes, the beads were washed 4 times with the cold ovary extract buffer with protease inhibitors, for 5 minutes each. For Western blotting, the beads were suspended in 30 μl of SDS loading buffer (50 mM Tris-HCl (pH 6.8), 100 mM DTT, 2% SDS, 0.1% bromophenol blue, 10% glycerol) and heated for 3 minutes at 95° C. before being loaded on a PAGE gel. Western blotting for Mtrm (FIG. 2B) was conducted by using anti-Mtrm antibody from guinea pigs and an Alkaline Phosphatase chromogen kit (BCIP/NBT) (Roche). Fluorescent Western blotting techniques were used to display both Mtrm and Polo from CO—IP on the same membrane.

Affinity Purification of Mtrm-Flag(3×) from Ovaries

In order to prepare a C-terminally 3×FLAG-tagged Mtrm for the MudPIT mass spectrometry assay, the UASP-mtrm-Flag (3×) construct was expressed in ovaries under the control of the nanos-GAL4 driver in a wild-type background. The extraction of protein from the ovaries was the same as described above. 100 μl of anti-FLAG beads were washed 2 times with pre-chilled 1×PBS and then 2 times with pre-chilled ovary extract buffer. The anti-FLAG beads were mixed with the extract supernatant, incubated and washed as described above. After washing, the beads bound with Mtrm-FLAG (3×) were finally transferred to a mini-column and washed with 25 ml of TBS (50 mM Tris-HCl, pH 7.4, 150 mM NaCl) at 4° C. When washing was completed, 300 μl TBS with 100 μg/ml 3×FLAG peptide was added to elute proteins. TCA was added to the eluted protein solution at a final concentration of 20%. The solution was mixed and kept on ice for at least 30 minutes. The solution was centrifuged at 14000 rpm at 4° C. for 15 minutes. The pellet was collected and 300 μl of pre-chilled acetone was gently added. After centrifuging again at 14000 rpm at 4° C. for 15 minutes, the pellet was carefully collected. The pellet was air dried and ready for the Mud PIT spectrometry analysis.

### Multidimensional Protein Identification Technology (MudPIT) Analysis

TCA-precipitated proteins were urea-denatured, reduced, alkylated and digested with endoproteinase Lys-C (Roche) followed by modified trypsin (Promega) as described in Washburn (2001) [1,5]. Peptide mixtures were loaded onto 100 μm fused silica microcapillary columns packed with 5-μm C₁₈ reverse phase (Aqua, Phenomenex), strong cation exchange particles (Partisphere SCX, Whatman), and reverse phase [42]. Loaded microcapillary columns were placed in line with a Quaternary 1100 series HPLC pump (±Agilent) and a LTQ linear ion trap mass spectrometer equipped with a nano-LC electrospray ionization source (ThermoFinnigan). Fully automated 10-step MudPIT runs were carried out on the electrosprayed peptides, as described in [43]. Tandem mass (MS/MS) spectra were interpreted using SEQUEST [44] against a database consisting of 17,348 Drosophila melanogaster proteins (non-redundant entries downloaded from NCBI, 2006 Nov. 28 release), and 177 usual contaminants (such as human keratins, IgGs, and proteolytic enzymes). To estimate false discovery rates (FDR), each non-redundant protein entry was randomized, keeping the same amino acid composition and length, doubling the search space to a total of 35,050 amino acid sequences (17,525 forward+17,525 shuffled sequences). Peptide/spectrum matches were selected and compared using DTASelect/CONTRAST [45] with the following criteria set: spectra/peptide matches were only retained if they had a DeltCn of at least 0.08, and a minimum XCorr of 1.8 for singly-, 2.0 for doubly-, and 3.0 for triply-charged spectra. In addition, peptides had to be fully-tryptic and at least 7 amino acids long. Combining all runs, proteins had to be detected by at least 2 such peptides or 1 peptide with 2 independent spectra. Under these criteria, the average FDR was 0.34±0. To estimate relative protein levels, Normalized Spectral Abundance Factors (NSAFs) were calculated for each non-redundant protein, as described in Zybailov (2006) and Paoletti (2006) [16,17]. Log-transformed NSAF values for proteins reproducibly detected in all three analyses were subjected to a two-tailed t-test to highlight proteins significantly enriched in the Mtrm purifications as opposed to negative controls as in Zybailov (2006) [17]. A differential modification search was set up to query a protein database containing only the sequences for Mtrm and Polo for peptides containing phosphorylated serines, threonines, tyrosines and oxidized methionines, i.e. SEQUEST “ASFP” (All Spectra against Few Proteins). The maximum number of modified amino acids per differential modification in a peptide was limited to four. After this search, an in-house developed script, sqt-merge [46] was used to combine the sets of SEQUEST output files (sqt files) generated from the normal “ASAP” search (All Spectra All Proteins, i.e. without modifications) and the phosphorylation “ASFP” search described above into one set. This merging step allowed only the best matches to be ranked first. The peptide matches contained in the merged sqt files were compiled and sorted using DTASelect [45]. For the third round of searches, spectra matching modified peptides were selected if they passed the conservative filtering criteria: minimum XCorr of 1.8 for +1, 2.0 for +2, and 3.0 for +3 spectra, with a maximum Sp rank of ten, and fully tryptic peptides with a minimum length of seven amino acids. Xcorr scores for isopeptides, in which any of several adjacent residues could be modified, tend to close resulting in low normalized differences in Xcorrs. The DeltaCn cut-off was hence set at 0.01 to allow such peptides to be further examined (“−m 0−t 0−Smn 7−y 2−s 10−2 2−3 3−d 0.01” DTASelect parameters). The coordinates for these spectra were written out into smaller ms2 files using the “—copy” utility of DTASelect. Because these subsetted ms2 files contained, at best, a few hundreds MS/MS spectra, they can be subjected to the same phosphorylation differential search against the complete Drosophila database (SEQUEST “MSAP”, Modified Spectra against All Proteins). This step allowed us to check that spectra matching modified peptides from Polo and Mtrm sequences did not find a better match against the larger protein database. Again, sqt-merge was used to bring together the results generated by these different searches. DTASelect was used to create reports listing all detected proteins and modified residues on Polo and Mtrm. All spectra matching modified peptides were visually assessed and given an evaluation flag (Y/M/N, for yes/maybe/no). The “no” matches were removed from the final data (−v 2 parameter in DTASelect). Results from different immunoprecipitations were compared using CONTRAST. NSAF5 (an in-house software by Tim Wen) was used to create the final report on all detected proteins across the different runs, calculate their respective NSAF values, and estimate false discovery rates (FDR). U_SPC6 software (in-house by Tim Wen) was used to extract total and modified spectral counts for each amino acid within the proteins of interest and calculate modification levels based on local spectral counts.

### Determining the Timing of NEB

To investigate the timing of NEB, 3 day old females were collected and fed on yeast for two days. Ovaries were dissected in halocarbon oil 700 (Sigma) on a slide and egg chambers were separated by mixing using a metal rod. Then, a coverslip was gently put on without pressing and mounting. After waiting for 20-30 minutes, the egg chambers were observed by phase contrast microscopy in dark view.

Examining Karyosome Structure Before and after NEB

To facilitate live imaging of the karyosome before and during NEB, stage 11-12 oocytes from well-fed females were dissected in halocarbon oil and then co-injected with Oli-Green Dye (Molecular Probes) to visualize DNA and Rhodamine-conjugated tubulin (Cytoskeleton) to visualize the spindle and to determine timing of the NEB. Oocytes with germinal vesicles were imaged using a LSM 510 META microscope (Zeiss). Images were acquired using the AIM software v 4 by taking a 10 series Z-stack at 1 micron intervals.

### In Situ Hybridization

The 1.686 satellite sequences (also known as the 359-bp repeats) on the X chromosome and AATAT repeats on the 4th chromosome were chosen as probes for in situ hybridization [29,30,47]. The 359-bp sequence of the 1.686 satellite sequences and (AATAT)₆ repeats were used for probe preparation. Alexa Fluor 488 dye was used for probes of 359-bp sequence on the X chromosome. For probes (AATAT)₆ on the 4th chromosome, Alexa Fluor 647 dye was used. The details of probe generation and labeling, egg chamber dissection and fixation, fluorescent in situ hybridization and microscopy observation were described previously [30]. In all oocytes examined for centromere co-orientation, 4^{th} chromosomes were observed as red masses of hybridization while the X chromosomes were observed as single bright green masses of hybridization. The FM7 balancer chromosome displays two green blocks of hybridization because of multiple inversions [30]. The AATAT probe is slightly hybridized with an X and FM7 balancer around the centromere region, and therefore both X and FM7 have a slight red signal at the centromere location.

### Matrimony Requires Two Evolutionarily Conserved Serines for Binding to Polo

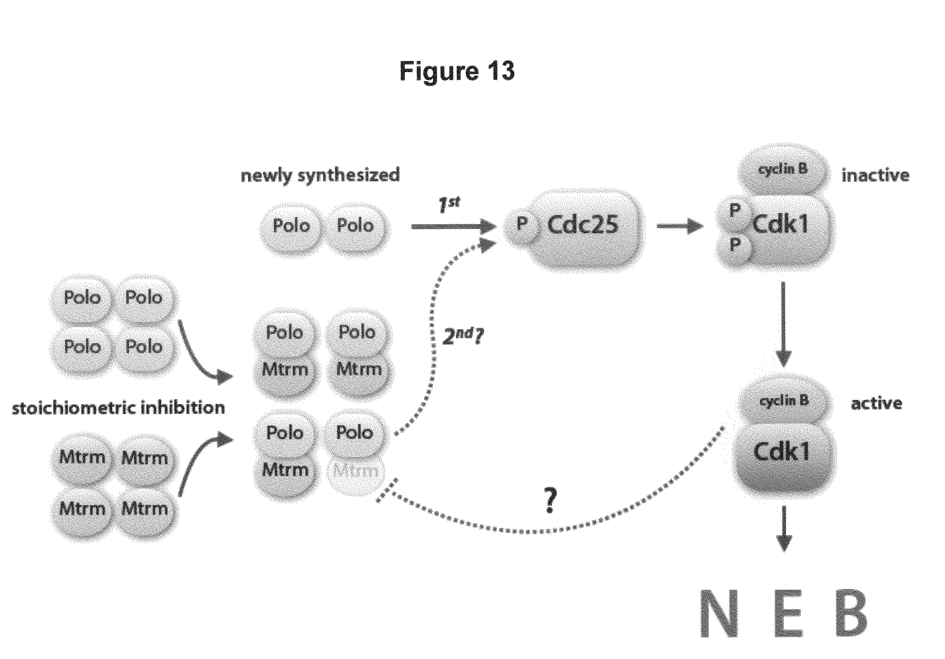

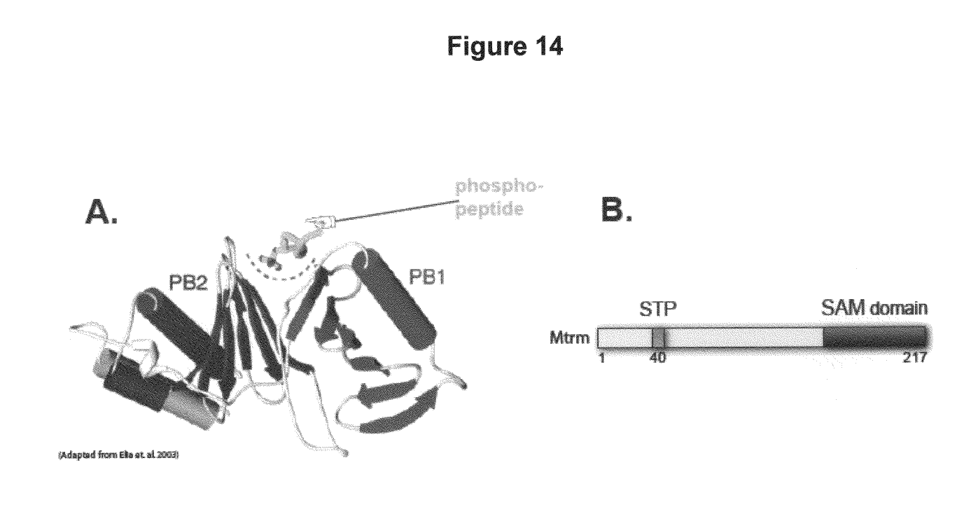

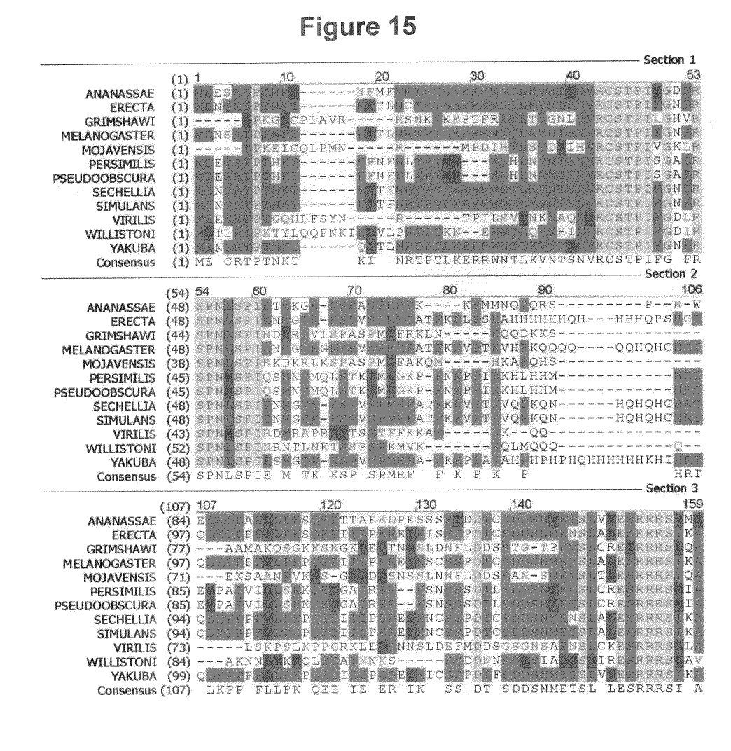

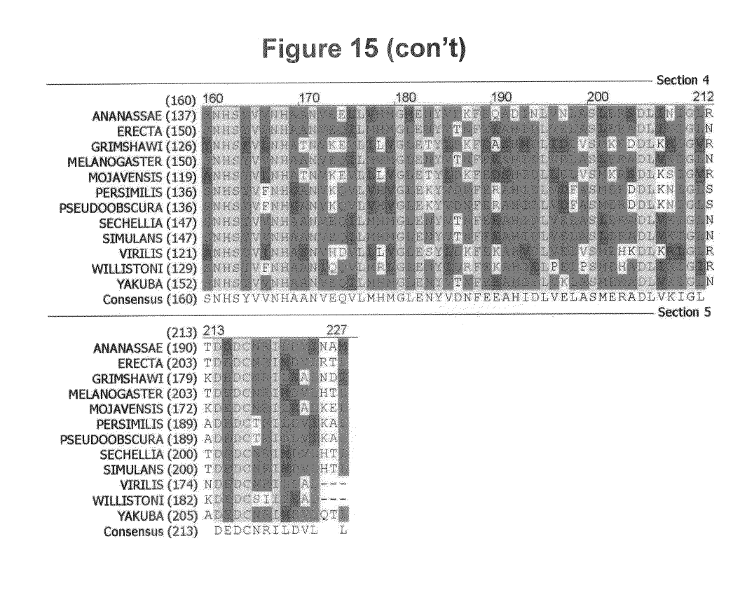

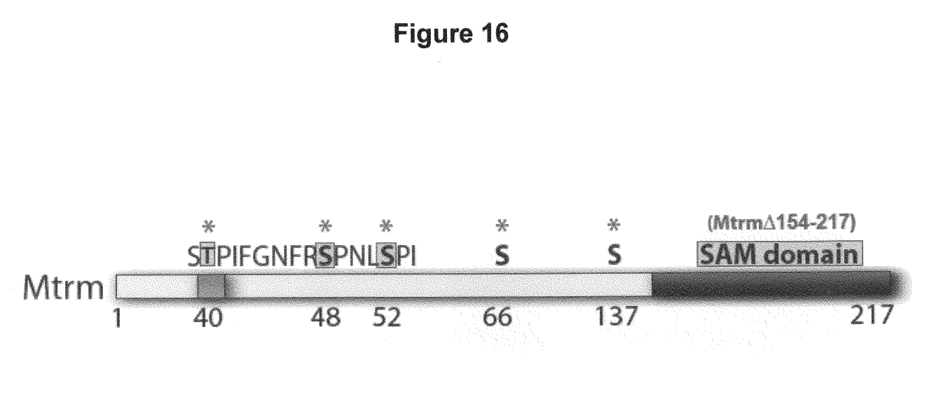

Female meiosis differs from other forms of cell division by the incorporation of two cell cycle arrests—the first of which occurs prior to the G2/M transition. In many organisms, Polo like kinase-1 (Plk1) has been implicated in the control of this first arrest to varying degrees; from acting as the “trigger” kinase that results in the activation of cyclin B-Cdk1 and subsequent nuclear envelope breakdown (NEB) to participating in the auto-amplification loop upon previous cyclin B-Cdk1 activation. Our work demonstrates that the regulation of this first meiotic arrest in Drosophila oocytes is also controlled by Polo (FIG. 13). Through a series of genetic, biochemical (FIG. 4D, E) and cytological analyses, we find Polo kinase to be regulated by a small inhibitory protein, Matrimony (Mtrm), during this phenomenal period of dormancy in female meiosis. Disruption of the careful balance of these two proteins in the oocyte results in meiotic defects including improper segregation of achiasmaste (nonexchange) homologous chromosomes and precocious NEB.1 Given Polo's key role in many cell cycle events, the question then arises as to how Mtrm precisely achieves such a feat. Recent work suggests that Mtrm inhibits Polo via direct binding to the Polo-box domain (PBD) of Polo (FIG. 14). We have shown previously that mutation of the central residue within a highly conserved PBD-binding consensus motif in Mtrm (MtrmT40) to alanine results in a complete loss of mtrm function. Here, both yeast two-hybrid (Y2H) analysis and site-specific mutagenesis of Drosophila transgenes suggest that two other evolutionarily conserved serines located near the PBD-binding motif (MtrmS48 and MtrmS52) are also critical for Mtrm function and direct binding to Polo.

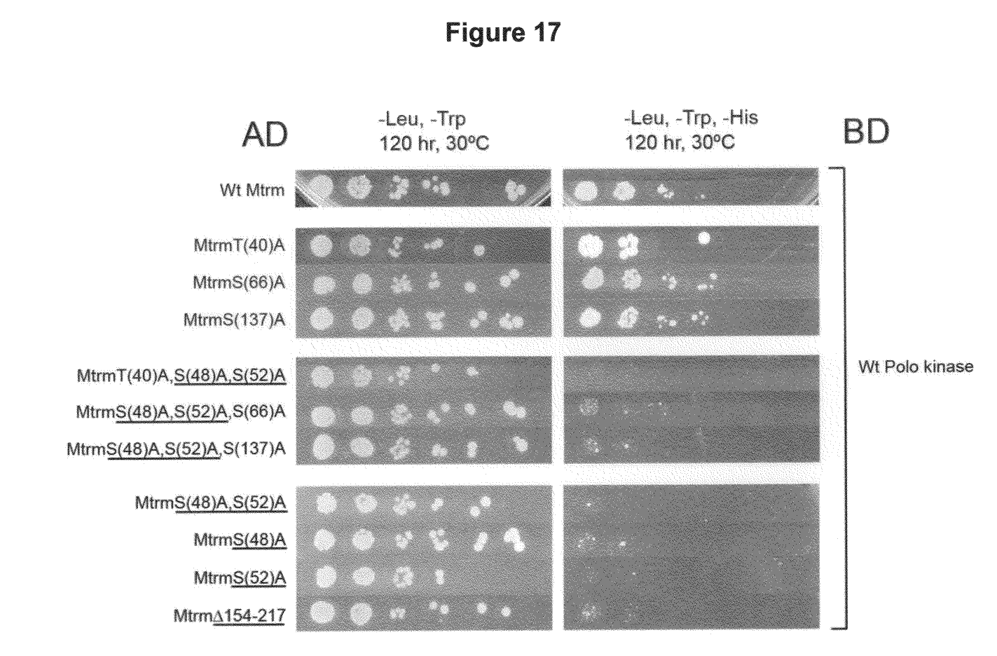

Our preliminary results indicate that in addition to MtrmT40, MtrmS48 and/or MtrmS52 are critical for Mtrm binding to Polo and for Mtrm function in Drosophila oocytes. We found that Mtrm mutants containing MtrmS48A and/or MtrmS52A ablated the interaction with Polo in the Y2H system (FIG. 17). We note that the MtrmT40A mutant only weakly bound Polo in the Y2H system, as evidenced by the growth of only 7 colonies following the first serial dilution and one colony at the third serial dilution (FIG. 17). Interestingly, the Y2H results correlate with an ongoing analysis of flies expressing mutant Mtrm proteins in a mtrm-compromised background.

Similar to the MtrmT40A mutant, we found that expression of mutants containing MtrmS48A and MtrmS52A does not rescue the defects in achiasmate (nonexchange) segregation (Table 1).

TABLE 1

| | N | % X NDJ | %4 NDJ |
|---|---|---|---|
| Wildtype(19) | 14,246 | .3 | .2 |
| mtrm^{[null]/+} | 1166 | 38.6 | 37.9 |
| P{mtrm^{[full-length]}}; mtrm^{[null]/+} | 1305 | 2.1 | 8.7 |
| P{mtrm^{[T40A]}}; mtrm^{[null]/+} | 970 | 43.7 | 36.9 |
| P{mtrm^{[T40A, S48A, S52A]}}; mtrm^{[null]/+} | 1472 | 40.1 | 40.4 |
| P{mtrm^{[S48A, S52A, S66A]}}; mtrm^{[null]/+} | 1262 | 55.9 | 45.7 |
| P{mtrm^{[S66A]}}; mtrm^{[null]/+} | 755 | 4.8 | 11.7 |

The highly conserved phosphorylatable residues, MtrmS48 and MtrmS52, in addition to the central residue of the PBD-binding site, MtrmT40, appear to be important for both the binding of Mtrm to Polo and the function of Mtrm in Drosophila. These observations call for further exploration and highlight important questions related to the preference of Polo's PBD to bind one PBD-binding site over another. Indeed, Mtrm contains one other putative PBD-binding motif (with the central residue: MtrmS124), however, previous work has demonstrated that site to be non-critical for Mtrm function and Mtrm-Polo binding (48).

It is worthy of note that MtrmS48 and MtrmS52 fall within a consensus motif for phosphorylation by GSK-3. It will be interesting to see whether GSK-3 phosphorylation at MtrmS48 is required for subsequent priming at MtrmT40, for sustained Polo PBD-binding, or for Mtrm degradation, as GSK-3 has been increasingly implicated in the process that mediates ubiquitin-mediated proteolysis.

Intriguingly, the Mtrm SAM domain appears to be important for Mtrm to efficiently bind Polo. Future work characterizing this C-terminal truncation in transgenic flies will provide further insight into this particular Y2H result.

### Protein Expression

mtrm and polo were cloned into pBacPAK8 with a Flag tag and 2XHA tag, respectively, at the N-terminus. The proteins were expressed using the BacPAK baculovirus expression system (Clontech) in Spodoptera frugiperda Sf9 cells. Sf9 cells were cultured at 27° C. in Sf-900 II SFM (Invitrogen) with 10% FBS. When cell density reached 1.5×10⁶/ml, the cells were infected with baculoviruses for 48 h. For single protein expression, baculoviruses containing either mtrm or polo was used to infect cells. For co-expression of Mtrm and Polo, two types of the baculoviruses were used together to infect cells. The cells were then harvested and lysed in buffer containing 20 mM HEPES pH 7.9, 1.5 mM MgCl₂, 100 mM NaCl; 0.2% Triton X-100 and 10% Glycerol with protease inhibitors. Cell lysates were ultra-centrifuged at 40,000 rpm for 40 min at 4° C. The supernatant was used for affinity purification.

### Affinity Purification

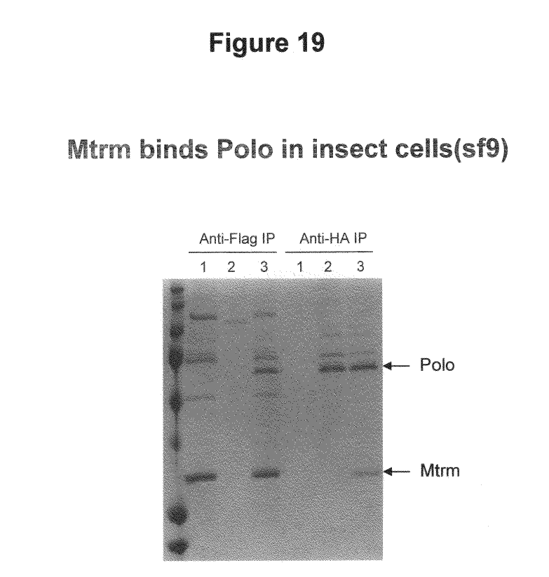

Anti-Flag and anti-HA agarose beads were obtained from Sigma. The agarose beads were pre-washed twice with 1×PBS and one wash with the above buffer. Anti-Flag and anti-HA affinity purifications were performed by incubating the prepared agarose beads with the lysates from Sf9 insect cells for 60 min with gently shaking at 4° C. After incubation, the agarose was washed 6 times with the above buffer for 6 min for each wash. After washing, the protein pulled down by anti-Flag was eluted using 200 μg/ml Flag peptide and the protein pulled down by anti-HA was eluted by 200 μg/ml 2×HA peptide. A part of each eluted protein sample was used for PAGE gel running. (FIG. 19).

### Protein In-Vitro Binding and Western Blotting

Protein from affinity-purified Flag-Mtrm and HA-Polo was used for an in vitro binding experiment. 50 μg Flag-Mtrm was mixed with the same amount of HA-Polo in 60 μl of the above buffer and incubated for 1 hr at 30° C. As a control, 50 μg HA-Polo in 60 μl was also incubated. After incubation, both protein samples were immunoprecipitated using 50 μl protein A agarose beads coated with anti-Mtrm antibody (from guinea pig) for 1 hr at 4° C. The agarose beads were washed 6 times with the above protein buffer.

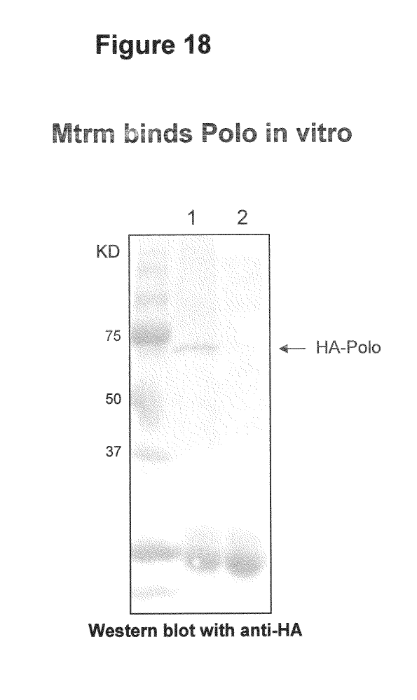

For Western blotting, the beads were suspended in 40 μl of SDS loading buffer (50 mM Tris-HCl (pH 6.8), 100 mM DTT, 2% SDS, 0.1% bromophenol blue, 10% glycerol) and heated for 3 min at 95° C. before being loaded on a PAGE gel. Western blotting for HA-Polo was conducted using anti-HA antibody from mouse and an Alkaline Phosphatase chromogen kit (BCIP/NBT) (Roche). (FIG. 18).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### CITED DOCUMENTS

The following documents, which have been cited above, are incorporated by reference as if recited in full herein:
1. King R C (1970) Ovarian Development in Drosophila melanogaster; King R C, editor: Academic Press, New York.
2. Mahowald A P, Kambysellis M P (1980) Oogenesis. In: Ashburner M, Wright T R, editors. Genetics and Biology of Drosophila, Vol 2-D. New York: Academic Press. pp. 141-224.
3. Riechmann V, Ephrussi A (2001) Axis formation during Drosophila oogenesis. Curr Opin Genet Dev 11: 374-383.
4. Page S L, Hawley R S (2001) c(3)G encodes a Drosophila synaptonemal complex protein. Genes Dev 15: 3130-3143.
5. Carpenter A T (1975) Electron microscopy of meiosis in Drosophila melanogaster females: II. The recombination nodule—a recombination-associated structure at pachytene? Proc Natl Acad Sci USA 72: 3186-3189.
6. Mirouse V, Formstecher E, Couderc J L (2006) Interaction between Polo and BicD proteins links oocyte determination and meiosis control in Drosophila. Development 133: 4005-4013.
7. Lee B H, Amon A (2003) Polo kinase—meiotic cell cycle coordinator. 1538-4101 (Print) 1538-4101 (Print). 400-402 p.
8. Lee B H, Amon A (2003) Role of Polo-like kinase CDC5 in programming meiosis I chromosome segregation. 1095-9203 (Electronic) 1095-9203 (Electronic). 482-486 p.
9. Harris D, Orme C, Kramer J, Namba L, Champion M, et al. (2003) A deficiency screen of the major autosomes identifies a gene (matrimony) that is haplo-insufficient for achiasmate segregation in Drosophila oocytes. Genetics 165: 637-652.
10. Formstecher E, Aresta S, Collura V, Hamburger A, Meil A, et al. (2005) Protein interaction mapping: a Drosophila case study. Genome Res. pp. 376-384.
11. Arbeitman M N, Furlong E E, Imam F, Johnson E, Null B H, et al. (2002) Gene expression during the life cycle of Drosophila melanogaster. Science 297: 2270-2275.
12. Clarke A S, Tang T T, Ooi D L, Orr-Weaver T L (2005) POLO kinase regulates the Drosophila centromere cohesion protein MEI-S332. Dev Cell 8: 53-64.
13. Moutinho-Santos T, Sampaio P, Amorim I, Costa M, Sunkel C E (1999) In vivo localization of the mitotic POLO kinase shows a highly dynamic association with the mitotic apparatus during early embryogenesis in Drosophila. Biol Cell 91: 585-596.
14. Llamazares S, Moreira A, Tavares A, Girdham C, Spruce B A, et al. (1991) polo encodes a protein kinase homolog required for mitosis in Drosophila. Genes Dev 5: 2153-2165.
15. Washburn M P, Wolters D, Yates J R, 3rd (2001) Large-scale analysis of the yeast proteome by multidimensional protein identification technology. Nat Biotechnol 19: 242-247.
16. Paoletti A C, Parmely T J, Tomomori-Sato C, Sato S, Zhu D, et al. (2006) Quantitative proteomic analysis of distinct mammalian Mediator complexes using normalized spectral abundance factors. Proc Natl Acad Sci USA 103: 18928-18933.
17. Zybailov B, Mosley A L, Sardiu M E, Coleman M K, Florens L, et al. (2006) Statistical analysis of membrane proteome expression changes in Saccharomyces cerevisiae. J Proteome Res 5: 2339-2347.
18. Elia A E, Cantley L C, Yaffe M B (2003) Proteomic screen finds pSer/pThr-binding domain localizing Plk1 to mitotic substrates. Science 299: 1228-1231.
19. Elia A E, Rellos P, Haire L F, Chao J W, Ivins F J, et al. (2003) The molecular basis for phosphodependent substrate targeting and regulation of Plks by the Polo-box domain. Cell 115: 83-95.
20. Lowery D M, Clauser K R, Hjerrild M, Lim D, Alexander J, et al. (2007) Proteomic screen defines the Polo-box domain interactome and identifies Rock2 as a Plk1 substrate. Embo J 26: 2262-2273.
21. Jang Y J, Ma S, Terada Y, Erikson R L (2002) Phosphorylation of threonine 210 and the role of serine 137 in the regulation of mammalian polo-like kinase. J Biol Chem 277: 44115-44120.
22. Davring L, Sunner M (1973) Female meiosis and embryonic mitosis in Drosophila melanogaster. I. Meiosis and fertilization. Hereditas 73: 51-64.
23. Mahowald A P, Tiefert M (1970) Fine structure changes in the Drosophila oocyte nucleus during a short period of RNA synthesis. Wilhelm Roux Arch 165: 8-25.
24. Nokkala S, Puro J (1976) Cytological evidence for a chromocenter in Drosophila melanogaster oocytes. Hereditas 83: 265-268.
25. Gilliland W D, Hughes S E, Cotitta J L, Takeo S, Xiang Y, et al. (2007) The Multiple Roles of Mps1 in Drosophila Female Meiosis. PLoS Genet. 3: e113.
26. Matthies H J, McDonald H B, Goldstein L S, Theurkauf W E (1996) Anastral meiotic spindle morphogenesis: role of the non-claret disjunctional kinesin-like protein. J Cell Biol 134: 455-464.
27. Skold H N, Komma D J, Endow S A (2005) Assembly pathway of the anastral Drosophila oocyte meiosis I spindle. J Cell Sci 118: 1745-1755.
28. Theurkauf W E, Hawley R S (1992) Meiotic spindle assembly in Drosophila females: behavior of nonexchange chromosomes and the effects of mutations in the nod kinesin-like protein. J Cell Biol 116: 1167-1180.
29. Dernburg A F, Sedat J W, Hawley R S (1996) Direct evidence of a role for heterochromatin in meiotic chromosome segregation. Cell 86: 135-146.
30. Xiang Y, Hawley R S (2006) The mechanism of secondary nondisjunction in Drosophila melanogaster females. Genetics 174: 67-78.
31. Morgan D O (2007) The Cell Cycle: Principles of Control (Primers in Biology) London, UK: New Science Press Ltd. 297 p.
32. Perdiguero E, Nebreda A R (2004) Regulation of Cdc25C activity during the meiotic G2/M transition. Cell Cycle 3: 733-737.
33. Chase D, Serafinas C, Ashcroft N, Kosinski M, Longo D, et al. (2000) The polo-like kinase PLK-1 is required for nuclear envelope breakdown and the completion of meiosis in Caenorhabditis elegans. Genesis 26: 26-41.
34. Qian Y W, Erikson E, Li C, Maller J L (1998) Activated polo-like kinase Plx1 is required at multiple points during mitosis in Xenopus laevis. Mol Cell Biol 18: 4262-4271.
35. Qian Y W, Erikson E, Maller J L (1999) Mitotic effects of a constitutively active mutant of the Xenopus polo-like kinase Plx1. Mol Cell Biol 19: 8625-8632.
36. Lenart P, Petronczki M, Steegmaier M, Di Fiore B, Lipp J J, et al. (2007) The small-molecule inhibitor BI 2536 reveals novel insights into mitotic roles of polo-like kinase 1. Curr Biol 17: 304-315.
37. Riparbelli M G, Callaini G, Glover D M (2000) Failure of pronuclear migration and repeated divisions of polar body nuclei associated with MTOC defects in polo eggs of Drosophila. J Cell Sci 113 (Pt 18): 3341-3350.
38. Blom N, Sicheritz-Ponten T, Gupta R, Gammeltoft S, Brunak S (2004) Prediction of post-translational glycosylation and phosphorylation of proteins from the amino acid sequence. Proteomics 4: 1633-1649.
39. Edgar B A, Sprenger F, Duronio R J, Leopold P, O'Farrell P H (1994) Distinct molecular mechanism regulate cell cycle timing at successive stages of Drosophila embryogenesis. Genes Dev 8: 440-452.
40. Archambault V, Zhao X, Carpenter A T, Glover D M (2007) Mutations in Drosophila Greatwall/Scant reveal its roles in mitosis and meiosis and suggest interdependence with Polo kinase. PLoS (in press). PLoS (in press).
41. Rorth P (1998) Gal4 in the Drosophila female germline. Mech Dev 78: 113-118.
42. McDonald W H, Ohi R, Miyamoto D T, Mitchison T J, Yates J R (2002) Comparison of three directly coupled HPLC MS/MS strategies for identification of proteins from complex mixtures: single-dimension LC-MS/MS, 2-phase MudPIT, and 3-phase MudPIT. International Journal of Mass Spectrometry 219: 245-251.
43. Florens L, Washburn M P (2006) Proteomic analysis by multidimensional protein identification technology. In: Nedelkov D, Nelson R W, editors. New and Emerging Proteomic Techniques: Humana Press. pp. 159-176.
44. Eng J K, McCormack A L, Yates J R (1994) An approach to correlate tandem mass spectral data of peptides with amino acid sequences in a protein database. Journal of the American Society for Mass Spectrometry 5: 976-989.
45. Tabb D L, McDonald W H, Yates J R, 3rd (2002) DTASelect and Contrast: tools for assembling and comparing protein identifications from shotgun proteomics. J Proteome Res 1: 21-26.
46. Zybailov B, Coleman M K, Florens L, Washburn M P (2005) Correlation of relative abundance ratios derived from peptide ion chromatograms and spectrum counting for quantitative proteomic analysis using stable isotope labeling. Anal Chem 77: 6218-6224.
47. Dernburg A F, Zalevsky J, Colaiacovo M P, Villeneuve A M (2000) Transgene-mediated cosuppression in the C. elegans germ line. Genes Dev 14: 1578-1583.
48. Xiang Y., et. al. (2007) PLoS Biol. December; 5(12): e323.
49. Smith S. K., et. al. (2008) Cell Cycle. March 15; 7(6), 698-701
50. Zitron, et. al. (1989) Genetics. 122, 801-821.

What is claimed is:

1. A method for modulating oocyte maturation comprising contacting an oocyte with an amount of a molecule selected from the group consisting of Polo kinase (Polo), an ortholog of Polo, a modulator of Polo or its ortholog, and combinations thereof, which amount is sufficient to achieve modulation of oocyte maturation.

2. The method according to claim 1, wherein the Polo ortholog is a human ortholog.

3. The method according to claim 1, wherein the modulator of Polo is a human ortholog of a Matrimony (Mtrm) polypeptide.

4. The method according to claim 1, wherein modulation of oocyte maturation comprises activating oocyte maturation.

5. The method according to claim 4, wherein activating oocyte maturation comprises contacting the oocyte with an amount of Polo or an ortholog thereof sufficient to initiate nuclear envelope breakdown.

6. The method according to claim 4, wherein activating oocyte maturation comprises contacting the oocyte with an amount of an inhibitor of Mtrm or an ortholog thereof, which is sufficient to initiate nuclear envelope breakdown.

7. The method according to claim 1, wherein modulation of oocyte maturation comprises inhibiting initiation of nuclear envelope breakdown.

8. The method according to claim 7, wherein inhibiting oocyte maturation comprises contacting the oocyte with an amount of Mtrm or an ortholog thereof sufficient to inhibit initiation of nuclear envelope breakdown.

9. The method according to claim 7, wherein inhibiting oocyte maturation comprises contacting the oocyte with an amount of an inhibitor of Polo or an ortholog thereof, which is sufficient to inhibit initiation of nuclear envelope breakdown.

10. The method according to claim 9, wherein the inhibitor is selected from the group consisting of HMN-214 ((E)-4-[2-[2-(p-methoxybenzenesulfonamide)-phenyl]ethenyl]pyridine-1-oxide, Nippon Shinyaku), ON-01910 (a small-molecule benzyl styryl sulfone polo-like kinase 1 inhibitor, Onconova), CYC800 (a small-molecule polo-like kinase-1 (Plk-1) inhibitor, Cyclacel), a signal inhibitor against Plk-1 (Rexahn), BI-2536 (a polo-like kinase 1 inhibitor, Boehringer Ingelheim), GSK-461364A (a thiophene amide polo-like kinase-1 (Plk) inhibitor, GlaxoSmithKline), PIKT inhibitors (Kiadis), PLK-1 inhibitors (Onconova), PLK-1 inhibitors (Sareum), and combinations thereof.

11. A method for identifying a candidate compound that modulates the binding of Matrimony or an ortholog thereof to Polo or an ortholog thereof, comprising the steps of:
(a) contacting Matrimony or an ortholog thereof with Polo or an ortholog thereof under conditions suitable to form a Matrimony-Polo complex;
(b) contacting the Matrimony-Polo complex with a candidate compound; and
(c) determining the ability of the candidate compound to modulate binding of Matrimony or an ortholog thereof to Polo or an ortholog thereof, wherein modulation of the binding of Matrimony or an ortholog thereof to Polo or an ortholog thereof indicates that the candidate compound is effective to modulate the binding of Matrimony or ortholog thereof to Polo or an ortholog thereof.

12. The method according to claim 11, wherein the candidate compound increases the binding of Matrimony or an ortholog thereof to Polo or an ortholog thereof.

13. The method of claim 11, wherein the candidate compound decreases the binding of Matrimony or an ortholog thereof to Polo or an ortholog thereof.

14. The method according to claim 11, wherein the candidate compound is selected from the group consisting of a nucleic acid, a polypeptide, a polysaccharide, a small organic or inorganic molecule, and combinations thereof.

15. The method according to claim 11, wherein the candidate compound is selected from the group consisting of a fusion protein, an antibody, an antibody mimetic, a domain antibody, a targeted aptamer, a RNAi, a siRNA, a shRNA, an antisense sequence, a small molecule, and combinations thereof.

16. The method according to claim 11, wherein the binding is determined using a method selected from the group consisting of a yeast two-hybrid (Y2H) assay, a fluorescence resonance energy transfer (FRET) assay, a bioluminescence resonance energy transfer (BRET) assay, a co-immunoprecipitation assay, a label transfer assay, a pull down assay, a tandem affinity purification (TAP) assay, an in vivo crosslinking assay, a chemical crosslinking assay, and a quantitative immunoprecipitation combined with knockdown (QUICK) assay.

17. The method according to claim 11, wherein the binding is determined using a yeast two-hybrid assay.

18. A method for identifying a functional ortholog of a Drosophila Matrimony polypeptide comprising:
(a) screening polypeptides from an oocyte preparation for their ability to interact with Polo kinase (Polo) or an ortholog thereof; and
(b) identifying which, if any, of the polypeptides screened in step (a) act as an inhibitor of Polo or an ortholog thereof.

19. The method according to claim 18, wherein the oocyte preparation is obtained from a human.

20. The method according to claim 18, wherein a polypeptide identified in step (b) comprises a Polo binding domain (PBD) having amino acids STP, SSP, or both STP and SSP.


SEQUENCE LISTING (13 sequences)

SEQ ID NO: 1 - DNA, 31 bp, Artificial
  Primer
ctcgaggatggccgcgaagcccgaggataag

SEQ ID NO: 2 - DNA, 35 bp, Artificial
  Primer
tctagattatgtgaacatcttctccagcattttcc

SEQ ID NO: 3 - DNA, 40 bp, Artificial
  primer pKpnI-mtrm-5
ggggtaccaaatggagaattctcgcacgcccacgaacaag

SEQ ID NO: 4 - DNA, 64 bp, Artificial
  primer mtrm-3-flag(1.5X)
gtccttgtagtccttgtcatcgtcgtccttgtagtcaagagtgtggagcacatccatgatacgg

SEQ ID NO: 5 - DNA, 78 bp, Artificial
  flag(3X)stop-Xbal primer
gctctagattacttgtcatcgtcgtccttgtagtccttgtcatcgtcgtccttgtagtccttgtcatcgtcgtccttg

SEQ ID NO: 6 - DNA, 46 bp, Artificial
  primer pmtrm-mut-ATG
cggggtaccaaaagatggagaattctcgcacgcccacgaacaagac

SEQ ID NO: 7 - DNA, 69 bp, Artificial
  primer pmtrm-STPre
gagattgggcgaacggaagttgccaaagatcggagcagagcatcgcacgttggaggtgttcaccttcag

SEQ ID NO: 8 - DNA, 69 bp, Artificial
  primer pmtrm-STP
ctgaaggtgaacacctccaacgtgcgatgctctgctccgatctttggcaacttccgttcgcccaatctc

SEQ ID NO: 9 - DNA, 43 bp, Artificial
  primer pmtrm-mut-TAA
gctctagattaaagagtgtggagcacatccatgatacgcttgc

SEQ ID NO: 10 - DNA, 50 bp, Artificial
  primer pmtrm-SSPre
ggtctccatattcgagtcatccgaacaggtatccggggcgctgcagctct

SEQ ID NO: 11 - DNA, 50 bp, Artificial
  primer pmtrm-SSP
agagctgcagcgccccggatacctgttcggatgactcgaatatggagacc

SEQ ID NO: 12 - DNA, 656 bp, Drosophila melanogaster
atggagaattctcgcacgcccacgaacaagaccaaaattacgcttaatcgcacgccaacgctaaaggagcgcagatggaacaccctgaaggtgaacacctccaacgtgcgatgctctactccgatctttggcaacttccgttcgcccaatctctcgcccatcgagaatatgggcacgaaggggaagagtccagtgtcgcccatgcggttcgctaccttcaagaaagtgccaacgaaggtgcatcccaagcagcagcagcagcagcagcatcagcactgccatcgcactcagtttaagcccccgccattcgtgctgcccaagccgcaggaggagatcatcgaaccggagcgagaaataaagagctgcagcagcccggatacctgttcggatgactcgaatatggagacctcactggcgttggagtcgcgtcgtcgttccatcaaagcatcgaaccactcgtacgtggttaaccatgccgccaatgtggaacagattctcatgcacatgggcctggagaactatgtgaccaatttcgaagaggctcacatcgatctggtggaactggcatccttggagcgtgctgatcttgttaaaatcggcctaaataccgatgaggattgcaaccgcatcatggatgtgctccacactctttaagt

SEQ ID NO: 13 - protein, 217 aa, Drosophila melanogaster
MENSRTPTNKTKITLNRTPTLKERRWNTLKVNTSNVRCSTPIFGNFRSPNLSPIENMGTKGKSPVSPMRFATFKKVPTKVHPKQQQQQQHQHCHRTQLKPPPFVLPKPQEEIIEPEREIKSCSSPDTCSDDSNMETSLALESRRRSIKASNHSYVVNHAANVEQILMHMGLENYVTNFEEAHIDLVELASLERADLVKIGLNTDEDCNRIMDVLHTL